(19) United States
(12) Patent Application Publication
(10) Pub. No.: US 2025/0108144 A1
(43) Pub. Date: Apr. 3, 2025

(54) VISCOELASTIC MATERIALS BASED ON MICROSTRUCTURED LIQUIDS

(71) Applicant: The Regents of the University of California, Oakland, CA (US)

(72) Inventors: Seunghyun Sim, Irvine, CA (US); Hyuna Jo, Irvine, CA (US)

(21) Appl. No.: 18/905,109

(22) Filed: Oct. 2, 2024

Related U.S. Application Data
(60) Provisional application No. 63/542,078, filed on Oct. 2, 2023.

(51) Int. Cl. A61L 26/00

(52) U.S. Cl. CPC A61L 26/008; A61L 26/0019; A61L 2300/208; A61L 2300/216

(57) ABSTRACT

The disclosure provides for viscoelastic materials based on microstructured liquids and applications thereof, including as underwater adhesives and wound care, and processes to make the viscoelastic materials.

### CROSS REFERENCE TO RELATED APPLICATIONS

This application claims priority under 35 U.S.C. § 119 from Provisional Application Ser. No. 63/542,078 filed Oct. 2, 2023, the disclosure of which is incorporated herein by reference.

### STATEMENT OF GOVERNMENT SUPPORT

This invention was made with Government support under Grant No. DMR-2011967, awarded by the National Science Foundation. The Government has certain rights in the invention.

### TECHNICAL FIELD

The disclosure provides for viscoelastic materials based on microstructured liquids, and applications thereof, including as underwater adhesives and wound care, and processes to make the viscoelastic materials.

### BACKGROUND

Underwater adhesives with fast-curing and robust adhesion are difficult to achieve due to surrounding water molecules hindering chemical bonding and reaction necessary for adhesion. Synthetic macromolecules (polymers) or organic solvents are commonly utilized for synthetic underwater adhesives to repel water molecules and increase adhesion to surfaces. However, challenges include elaborate and expensive multi-step chemical synthesis for polymer species and potential toxicity from organic solvents. The transition from a lab scale to a large industrial production scale remains difficult due to these reasons. In addition, most polymer-based adhesives to date require long curing times (>1 h), which might pose difficulties in applications requiring rapid performance, such as emergency wound closure or repairs.

### SUMMARY

Functionality in biological materials arises from complex hierarchical structures formed through self-assembly processes. Reported herein is a kinetically trapped self-assembly of an elastic network of liquid droplets, and its utility for tough and fast-acting adhesive. This complex structure was made from a one-pot mixture of scalable small molecule precursors. Liquid-liquid phase separation accompanied by silanol hydrolysis, condensation, and zwitterionic self-association yielded a viscoelastic solid with interconnected liquid droplets. These hierarchical microstructures increased toughness and enabled underwater adhesion for a range of substrates.

Accordingly, the disclosure provides compositions and processes for preparing viscoelastic materials that are tough and act rapidly. In particular embodiment, the processes used to make a viscoelastic material of the disclosure, comprises mixing an aqueous solution comprising silyl methacrylate-based compounds with zwitterion methacrylate-based compounds, and through hydrolysis, condensation, liquid-liquid phase separation, and zwitterionic interaction, a homogenous mixture of non-viscous liquid spontaneously develops into a sticky viscoelastic material. The compositions and processes of the disclosure overcome major limitations of other polymer-based adhesives by using two scalable small molecule precursors instead of multi-step synthesis of designer polymers. The processes disclosed herein can be scaled up to a commercial scale and the resulting viscoelastic materials can find use in many industries. For example, the viscoelastic materials can be used in medical (e.g., adhesive for dressing and bandages) and watercraft industries (e.g., underwater adhesive). For underwater adhesive applications, a photoinitator can be added to sticky viscoelastic material, and the resulting adhesive can be applied to surfaces underwater, and then fixed by photocuring the adhesive in mere minutes. Due to the viscoelastic material disclosed herein having an elastic network of compartmentalized liquid droplets, surface adhesion, stable deposition underwater, and toughness can be all realized. Accordingly, the disclosure provides a platform for robust adhesives for rapid underwater repair or emergency wound care.

In a particular embodiment, the disclosure provides for a viscoelastic material having a solid or gel-like consistency, comprising: an elastic network of compartmentalized liquid droplets, wherein each liquid droplet has an exterior surface and inner compartment, wherein the inner compartment comprises a silyl-based compound and wherein the exterior surface comprises a zwitterion-based compound, wherein the electrostatic interactions between the compartmentalized liquid droplets interlock the compartmentalized liquid droplets together to form a viscoelastic material having a solid or gel-like consistency. In another embodiment, the elastic network of compartmentalized liquid droplets is a kinetic product. In yet another embodiment, the silyl-based compound forms interconnected silyl-based compounds via siloxane bonds. In a further embodiment, the silyl-based compound is selected from 3-(trimethoxysilyl)propyl methacrylate, 3-(trimethoxysilyl)methyl methacrylate, 3-(trimethoxysilyl)ethyl methacrylate, 3-(trimethoxysilyl)butyl methacrylate, 3-(dimethoxy(methyl)silyl)propyl methacrylate, 3-(dimethoxy(methyl)silyl)methyl methacrylate, 3-(dimethoxy(methyl)silyl)ethyl methacrylate, 3-(dimethoxy(methyl)silyl)butyl methacrylate, 3-(dimethyl(methoxy)silyl)propyl methacrylate, 3-(dimethyl(methoxy)silyl)methyl methacrylate, 3-(dimethyl(methoxy)silyl)ethyl methacrylate, 3-(dimethyl(methoxy)silyl)butyl methacrylate, (trimethylsilyl)methacrylate, 3-[Tris(trimethylsiloxy)silyl]propyl methacrylate, bis(trimethylsiloxy)methylsilyl methacrylate, triisopropylsilyl methacrylate, and tributylsilyl methacrylate. In yet a further embodiment, the silyl-based compound is 3-(trimethoxysilyl)propyl methacrylate (TMeOSMA). In another embodiment, the zwitterion-based compound is selected from 2-(methacryloyloxy)ethyl]dimethyl-(3-sulfopropyl)ammonium hydroxide, 2-(methacryloyloxy)ethyl]dimethyl-(3-sulfoethyl)ammonium hydroxide, 2-(methacryloyloxy)ethyl]dimethyl-(3-sulfobutyl)ammonium hydroxide, 3-3-[[2-(Methacryloyloxy)ethyl]dimethylammonio]propionate, 3-3-[[2-(Methacryloyloxy)ethyl]dimethylammonio]acetate, 3-3-[[2-(Methacryloyloxy)ethyl]dimethylammonio]butyrate, and 2-methacryloyloxyethyl phosphorylcholine. In yet another embodiment, the zwitterion-based polymerizable compound is 2-(methacryloyloxy)ethyl]dimethyl-(3-sulfopropyl)ammonium hydroxide (sulfobetaine methacrylate, SBMA). In a further embodiment, the ratio of the molality concentration of the zwitterion-based compound to the molality concentration of the silyl-based compound is from 2:1 to 50:1. In yet a further embodiment, the ratio of the molality concentration of the zwitterion-based compound to the molality concentration of the silyl-based compound is from 4:1 to 20:1. In another embodiment, the ratio of the molality concentration of the zwitterion-based compound to the molality concentration of the silyl-based compound is about 5:1. In yet another embodiment, the viscoelastic material further comprises a reactive monomer. In a further embodiment, the reactive monomer is methacrylic acid N-hydroxysuccinimide ester (NHSMA), methacrylic acid N-hydroxysuccinimide ester, N-(Hydroxymethyl)acrylamide acrylic acid, N-hydroxysuccinimide ester paraformaldehyde, glycidyl methacrlyate, and glycidyl acrylate. In yet a further embodiment, the viscoelastic material further comprises a photoinitiator. In another embodiment, the photoinitiator is selected from lithium phenyl-2,4,6-trimethylbenzoylphosphinate, sodium 4-[2-(4-morpholino)benzoyl-2-dimethylamino]butylbenzenesulphone (MBS), 2-hydroxy-1-[4-(2-hydroxyethoxy) phenyl]-2-methyl-1-propanone, BAPO-OLi, BAPO-ONa, and 2,2′-azobis[2-methyl-N-(2-hydroxyethyl) promionamide] (VA-086), isopropyl thioxanthone (ITX)-ethyl 4-(dimethylamino)benzoate, acetophenone, anisoin, anthraquinone, anthraquinone-2-sulfonic acid, (benzene) tricarbonylchromium, benzil, benzoin, benzoin ethyl ether, benzoin isobutyl ether, benzoin methyl ether, benzophenone, benzophenone/1-hydroxycyclohexyl phenyl ketone (50/50), 3,3′,4,4′-benzophenonetetracarboxylic dianhydride, 4-benzoylbiphenyl, 2-benzyl-2-(dimethylamino)-4′morpholinobutyrophenone, 4,4′-bis(diethylamino)benzophenone, camphorquinon, 2-chlorothioxanthen-9-one, (cumene)cyclopentadienyliron(II) hexafluorophosphate, dibenzosuberenone, 2,2-diethoxyacetophenone, 4,4′-dihydroxybenzophenone, 2,2-dimethoxy-2-phenylacetophenone, 4-(dimethylamino)benzophenone, 4,4′-dimethylbenzil, 2,5-dimethylbenzophenone, 3,4-dimethylbenzophenone, diphenyl(2,4,6-trimethylbenzoyl)phosphine oxide/2-hydroxy-2-methylpropiophenone (50/50), 4′-ethoxyacetophenone, 2-ethylanthraquinone, ferrocene, 3′-hydroxyacetophenone, 4′-hydroxyacetophenone, 3-hydroxybenzophenone, 4-hydroxybenzophenone, 1-hydroxycyclohexyl phenyl ketone, 2-hydroxy-2-methylpropiophenone, 2-methylbenzophenone, 3-methylbenzophenone, methybenzoylformate, 2-methyl-4′-(methylthio)-2-morpholinopropiophenone, phenanthrenequinone, 4′-phenoxyacetophenone, thioxanthen-9-one, triarylsulfonium hexafluoroantimonate salts and triarylsulfonium hexafluorophosphate salts. In yet another embodiment, the photoinitiator is a water soluble photoinitiator selected from lithium phenyl-2,4,6-trimethylbenzoylphosphinate (LAP), sodium 4-[2-(4-morpholino)benzoyl-2-dimethylamino]butylbenzenesulphone (MBS), 2-hydroxy-1-[4-(2-hydroxyethoxy) phenyl]-2-methyl-1-propanone, BAPO-OLi, BAPO-ONa, and 2,2′-azobis[2-methyl-N-(2-hydroxyethyl) promionamide] (VA-086). In a certain embodiment, the water soluble photoinitiator is LAP. In another embodiment, the viscoelastic material comprises LAP a final concentration from 2 mM to 4 mM.

In a particular embodiment, the disclosure provides for an adhesive comprising a viscoelastic material of the disclosure. In another embodiment, the adhesive is capable of adhering to a substrate that is exposed to an aqueous environment. In a further embodiment, the aqueous environment comprises water that comprises from 0 ppt up to 50 parts per thousand of dissolved salts. In yet a further embodiment, the aqueous environment is fresh water, brackish water, saline, seawater, or brine.

In a certain embodiment, the disclosure also provides a bandage, dressing, or medical implant comprising a viscoelastic material of the disclosure. In another embodiment, the viscoelastic material is capable of adhering to biological tissue. In yet a further embodiment, the biological tissue is selected from skin tissue, hair tissue, nail tissue, corneal tissue, tongue tissue, oral cavity tissue, esophageal tissue, anal tissue, urethral tissue, vaginal tissue, urinary epithelial tissue, salivary gland tissue, mammary gland tissue, lacrimal gland tissue, sweat gland tissue, prostate gland tissue, bulbourethral gland tissue, Bartholin's gland tissue, uterine tissue, respiratory and gastrointestinal tract goblet cell tissue, gastric mucosal tissue, gastric gland tissue, pancreatic tissue, pulmonary tissue, pituitary gland tissue, thyroid gland tissue, parathyroid gland tissue, testicular tissue, ovarian tissue, respiratory gland tissue, gastrointestinal gland tissue, adrenal gland tissue, renal tissue, liver tissue, adipose tissue, duct cell tissue, gall bladder tissue, epidydimal tissue, vas deferens tissue, blood vessel tissue, lymph gland tissue, lymphatic duct tissue, synovial tissue, serosal tissue, squamous tissue, cochlear tissue, choroid plexus tissue, ependymal tissue, dural tissue, pia-arachnoid tissue, sclera tissue, retinal tissue, iris tissue, ciliary tissue, dental tissue, otic tissue, ligament tissue, tendon tissue, elastic cartilage tissue, fibrocartilage tissue, hyaline cartilage tissue, bone marrow tissue, intervertebral disc tissue, compact bone tissue, cancellous bone tissue, skeletal muscle tissue, cardiac muscle tissue, smooth muscle tissue, cardiac valve tissue, pericardial tissue, pleural tissue, peritoneal tissue, blood cell tissue, neuronal tissue, glial tissue, sensory transducer cell tissue, pain sensitive tissue, autonomic neuron tissue, peripheral nervous system tissue, cranial nerve tissue, ocular lens tissue, germ cell tissue, thymus tissue, placental tissue, fetal membrane tissue, umbilical tissue, stem cell tissue, mesodermal tissue, ectodermal tissue, endodermal tissue, autologous tissue, allograft tissue or a combination thereof. In another embodiment, the biological tissue is skin tissue.

In a particular embodiment, the disclosure provides a process of manufacturing a viscoelastic material disclosed herein, comprising: forming a mixture by combining a silyl-based compound and an acid, to an aqueous solution comprising a zwitterion-based compound; vigorously introducing a gas into the mixture to form a homogenous mixture; optionally, adding a photoinitator and/or a reactive monomer to the homogenous mixture; solidifying the homogenous mixture into a viscoelastic material having a solid or gel-like consistency, wherein the homogenous mixture spontaneously forms the viscoelastic material over a period of time. In a further embodiment, the mixture further comprises a salt. In yet a further embodiment, the salt is selected from sodium chloride, potassium chloride, and calcium chloride. In yet a further embodiment, the mixture comprises the salt at a molality from 0 m to 10 m. In a certain embodiment, the mixture has a pH from 1 to 6. In another embodiment, the mixture has a pH from 1 to 4. In a further embodiment, the silyl-based compound and/or the zwitterion-based compound comprises a polymerizable moiety selected from methacrylate, acrylate, methacrylamide, and acrylamide. IN another embodiment, the silyl-based compound is selected from 3-(trimethoxysilyl)propyl methacrylate, 3-(trimethoxysilyl)methyl methacrylate, 3-(trimethoxysilyl)ethyl methacrylate, 3-(trimethoxysilyl)butyl methacrylate, 3-(dimethoxy(methyl)silyl)propyl methacrylate, 3-(dimethoxy(methyl)silyl)methyl methacrylate, 3-(dimethoxy(methyl)silyl)ethyl methacrylate, 3-(dimethoxy(methyl)silyl)butyl methacrylate, 3-(dimethyl(methoxy)silyl)propyl methacrylate, 3-(dimethyl(methoxy)silyl)methyl methacrylate, 3-(dimethyl(methoxy)silyl)ethyl methacrylate, 3-(dimethyl(methoxy)silyl)butyl methacrylate, (trimethylsilyl)methacrylate, 3-[Tris(trimethylsiloxy)silyl]propyl methacrylate, bis(trimethylsiloxy)methylsilyl methacrylate, triisopropylsilyl methacrylate, and/or tributylsilyl methacrylate. In a certain embodiment, the silyl methacrylate-based compound is 3-(trimethoxysilyl)propyl methacrylate. In a further embodiment, the zwitterion methacrylate-based compound is selected from 2-(methacryloyloxy)ethyl]dimethyl-(3-sulfopropyl)ammonium hydroxide, 2-(methacryloyloxy)ethyl]dimethyl-(3-sulfoethyl)ammonium hydroxide, 2-(methacryloyloxy)ethyl]dimethyl-(3-sulfobutyl)ammonium hydroxide, 3-3-[[2-(Methacryloyloxy)ethyl]dimethylammonio]propionate, 3-3-[[2-(Methacryloyloxy)ethyl]dimethylammonio]acetate, 3-3-[[2-(Methacryloyloxy)ethyl]dimethylammonio]butyrate, and 2-methacryloyloxyethyl phosphorylcholine. In yet a further embodiment, the zwitterion methacrylate-based compound is 2-(methacryloyloxy)ethyl]dimethyl-(3-sulfopropyl)ammonium hydroxide. In a certain embodiment, the acid is hydrochloric acid or sulfuric acid. In yet another embodiment, the gas that is bubbled through the mixture is selected from air, nitrogen gas, helium gas, argon gas, or carbon dioxide. In certain embodiment, the gas that is bubbled through the acidic aqueous solution is nitrogen gas. In a further embodiment, the homogenous mixture comprises the reactive monomer. In yet a further embodiment, the reactive monomer is methyacrylic acid N-hydroxysuccinimide ester, methacrylic acid N-hydroxysuccinimide ester, N-(Hydroxymethyl)acrylamide acrylic acid, N-hydroxysuccinimide ester paraformaldehyde, glycidyl methacrlyate, and glycidyl acrylate. In another embodiment, the homogenous mixture comprises the photoinitator. In a further embodiment, the photoinitator is selected from lithium phenyl-2,4,6-trimethylbenzoylphosphinate, sodium 4-[2-(4-morpholino)benzoyl-2-dimethylamino]butylbenzenesulphone (MBS), 2-hydroxy-1-[4-(2-hydroxyethoxy) phenyl]-2-methyl-1-propanone, BAPO-OLi, BAPO-ONa, and 2,2′-azobis[2-methyl-N-(2-hydroxyethyl) promionamide] (VA-086), isopropyl thioxanthone (ITX)-ethyl 4-(dimethylamino)benzoate, acetophenone, anisoin, anthraquinone, anthraquinone-2-sulfonic acid, (benzene) tricarbonylchromium, benzil, benzoin, benzoin ethyl ether, benzoin isobutyl ether, benzoin methyl ether, benzophenone, benzophenone/1-hydroxycyclohexyl phenyl ketone (50/50), 3,3′,4,4′-benzophenonetetracarboxylic dianhydride, 4-benzoylbiphenyl, 2-benzyl-2-(dimethylamino)-4′morpholinobutyrophenone, 4,4′-bis(diethylamino)benzophenone, camphorquinon, 2-chlorothioxanthen-9-one, (cumene)cyclopentadienyliron(II) hexafluorophosphate, dibenzosuberenone, 2,2-diethoxyacetophenone, 4,4′-dihydroxybenzophenone, 2,2-dimethoxy-2-phenylacetophenone, 4-(dimethylamino)benzophenone, 4,4′-dimethylbenzil, 2,5-dimethylbenzophenone, 3,4-dimethylbenzophenone, diphenyl(2,4,6-trimethylbenzoyl)phosphine oxide/2-hydroxy-2-methylpropiophenone (50/50), 4′-ethoxyacetophenone, 2-ethylanthraquinone, ferrocene, 3′-hydroxyacetophenone, 4′-hydroxyacetophenone, 3-hydroxybenzophenone, 4-hydroxybenzophenone, 1-hydroxycyclohexyl phenyl ketone, 2-hydroxy-2-methylpropiophenone, 2-methylbenzophenone, 3-methylbenzophenone, methybenzoylformate, 2-methyl-4′-(methylthio)-2-morpholinopropiophenone, phenanthrenequinone, 4′-phenoxyacetophenone, thioxanthen-9-one, triarylsulfonium hexafluoroantimonate salts and triarylsulfonium hexafluorophosphate salts. In another embodiment, the photoinitiator is a water soluble photoinitiator selected from lithium phenyl-2,4,6-trimethylbenzoylphosphinate (LAP), sodium 4-[2-(4-morpholino)benzoyl-2-dimethylamino]butylbenzenesulphone (MBS), 2-hydroxy-1-[4-(2-hydroxyethoxy) phenyl]-2-methyl-1-propanone, BAPO-OLi, BAPO-ONa, and 2,2′-azobis[2-methyl-N-(2-hydroxyethyl) promionamide] (VA-086). In a certain embodiment, the photoinitiator is LAP. In another embodiment, the process further comprises the step of: curing or fixing the viscoelastic material by exposing the viscoelastic material to light having a wavelength in the ultraviolet-visible spectral range.

In a particular embodiment, the disclosure provides a process of manufacturing a viscoelastic material disclosed herein, comprising: forming a mixture by combining a silyl-based compound and an acid, to an aqueous solution comprising a zwitterion-based compound; vigorously introducing a gas into the mixture to form a homogenous mixture; optionally, adding a photoinitator and/or a reactive monomer to the homogenous mixture; solidifying the homogenous mixture into a viscoelastic material having a solid or gel-like consistency, wherein the homogenous mixture spontaneously forms the viscoelastic material over a period of time. In another embodiment, the silyl-based compound is 3-(trimethoxysilyl)propyl methacrylate (TMeOSMA); the zwitterion-based compound is 2-(methacryloyloxy)ethyl]dimethyl-(3-sulfopropyl)ammonium hydroxide (SBMA); the pH of the mixture is from 1 to 4; and/or the gas is nitrogen gas. In yet another embodiment, the homogenous mixture comprises the photoiniator, or comprises the photoiniator and the reactive monomer, and the method further comprises the step of: curing or fixing the viscoelastic material by exposing the viscoelastic material to light having a wavelength in the ultraviolet-visible spectral range, wherein the photoiniator is lithium phenyl-2,4,6-trimethylbenzoylphosphinate (LAP) and the reactive monomer is methacrylic acid N-hydroxysuccinimide ester (NHSMA).

In a particular embodiment, the disclosure provides for a viscoelastic material made by a manufacturing process as substantially disclosed herein.

In a certain embodiment, the disclosure also provides for an adhesive comprising a viscoelastic material made by a process as substantially disclosed herein. In a further embodiment, the adhesive is capable of adhering to a substrate that is exposed to an aqueous environment. In yet a further embodiment, the aqueous environment comprises water that comprises from 0 ppt up to 50 parts per thousand of dissolved salts. In another embodiment, the aqueous environment is fresh water, brackish water, saline, seawater, or brine.

In a particular embodiment, the disclosure provides for a bandage, dressing, or medical implant comprising an adhesive comprising a viscoelastic material made by a process as substantially disclosed herein. In a further embodiment, the adhesive is capable of adhering to biological tissue. In another embodiment, the biological tissue is selected from skin tissue, hair tissue, nail tissue, corneal tissue, tongue tissue, oral cavity tissue, esophageal tissue, anal tissue, urethral tissue, vaginal tissue, urinary epithelial tissue, salivary gland tissue, mammary gland tissue, lacrimal gland tissue, sweat gland tissue, prostate gland tissue, bulbourethral gland tissue, Bartholin's gland tissue, uterine tissue, respiratory and gastrointestinal tract goblet cell tissue, gastric mucosal tissue, gastric gland tissue, pancreatic tissue, pulmonary tissue, pituitary gland tissue, thyroid gland tissue, parathyroid gland tissue, testicular tissue, ovarian tissue, respiratory gland tissue, gastrointestinal gland tissue, adrenal gland tissue, renal tissue, liver tissue, adipose tissue, duct cell tissue, gall bladder tissue, epidydimal tissue, vas deferens tissue, blood vessel tissue, lymph gland tissue, lymphatic duct tissue, synovial tissue, serosal tissue, squamous tissue, cochlear tissue, choroid plexus tissue, ependymal tissue, dural tissue, pia-arachnoid tissue, sclera tissue, retinal tissue, iris tissue, ciliary tissue, dental tissue, otic tissue, ligament tissue, tendon tissue, elastic cartilage tissue, fibrocartilage tissue, hyaline cartilage tissue, bone marrow tissue, intervertebral disc tissue, compact bone tissue, cancellous bone tissue, skeletal muscle tissue, cardiac muscle tissue, smooth muscle tissue, cardiac valve tissue, pericardial tissue, pleural tissue, peritoneal tissue, blood cell tissue, neuronal tissue, glial tissue, sensory transducer cell tissue, pain sensitive tissue, autonomic neuron tissue, peripheral nervous system tissue, cranial nerve tissue, ocular lens tissue, germ cell tissue, thymus tissue, placental tissue, fetal membrane tissue, umbilical tissue, stem cell tissue, mesodermal tissue, ectodermal tissue, endodermal tissue, autologous tissue, allograft tissue or a combination thereof. In yet another embodiment, the biological tissue is skin tissue.

The details of one or more embodiments of the disclosure are set forth in the accompanying drawings and the description below. Other features, objects, and advantages will be apparent from the description and drawings, and from the claims.

### DESCRIPTION OF DRAWINGS

FIG. 1A-D provides a schematic illustration of embodiments of the compositions and methods of the disclosure. (A) Typical LLPS leads to macroscopic segregation of two distinct phases (left). Previous approaches on constructing materials bearing complex liquid structures involve trapping liquid droplets within hydrogel scaffold or assembling stabilized droplets (right). (B) Summary and features of this work. (C) Chemical structures of the two small molecule precursors in this study. (D) A schematic illustration of the compartmentalized network formation over time.

FIG. 2A-C presents optical images of the mixtures containing SBMA and TMeOSMA (ratio=5, total concentration=5.7 m) treated with (A) air bubbling, (B) vortexing, (C) sonication instead of N₂ bubbling, and their resulting products after 16 hours.

FIG. 3A-D presents the formation of an elastic network of compartments via LLPS. (A) Representative optical microscopy and photographic images showing the network formation process. Scale bars=50 m. (B) Storage moduli G′ and loss moduli G″ of the mixture containing SBMA and TMeOSMA over time. (C, D) Fluorescence microscopy (kex=495 nm; λ_{obs}=690-740 nm) of the mixture containing (C) FAM-TMeOSMA and (D) FAM-SBMA. Images were taken at 30 min assembly time after mixing and N₂ bubbling. Scale bars=20 μm.

FIG. 4 presents a photographic image (left) and a representative phase contrast microscope image (right) of the mixture of SBMA/TMeOSMA at 80° C. for 10 min. Scale bar=30 μm.

FIG. 5A-F demonstrates hydrolysis and silanol condensation occur within liquid droplets. (A)²⁹Si NMR spectroscopy of the mixture containing TMeOSMA and SBMA over time, and chemical structure illustrating different states of silicon atoms. (B) Degree of condensation (%) of the mixture containing TMeOSMA and SBMA as a function of reaction time. (C) FT-IR spectroscopy of the mixture containing TMeOSMA and SBMA over time. (D, E) Time-lapse confocal laser scanning microscopy images (λₑₓ=515 nm; λ_{obs}=550-630 nm) of the mixture containing Nile red dye (2 μM) at the assembly times of (D) 30 min and (E) 8 hours after photobleaching (λₑₓ=515 nm, 100% intensity). Scale bars=30 m. (F) Diffusion coefficient of the mixture of TMeOSMA and SBMA at different assembly times calculated from the FRAP experiments. Error bars represent ±s.e.m. N=3.

FIG. 6 provides confocal laser scanning microscopy images (λₑₓ=515 nm, λₑₘ=550-630 nm) of the mixtures of SBMA/TMeOSMA containing Nile red (2 μM) assembled for 30 min, 4 h, 8 h, and 16 h. The center of the droplet was photobleached (λₑₓ=515 nm, 100% intensity), and the recovery patterns were recorded. Scale bars=30 m.

FIG. 7 provides the normalized fluorescence intensity of the bleached area in FIG. 6.

FIG. 8A-H shows the effect of molecular structures and chemical environment on the END phase formation. (A) The chemical structure of different zwitterions: sulfobetaine methacrylate (SBMA), carboxybetaine methacrylate (CBMA), and phosphorylcholine methacrylate (PCMA). (B) Schematic illustration of the products from mixing TMeOSMA and a zwitterionic precursor. Macroscopic single phase represents a homogeneous mixture (liquid or gel state), and macroscopic two phases represent macroscopic phase segregation of two distinct layers (liquid or gel state). (C-E) Phase diagrams of TMeOSMA mixtures with (C) SBMA, (F) CBMA, and (G) PCMA, varying the total concentration of methacrylate species and zwitterion ratio. (F) Representative optical microscopy images of the mixtures containing SBMA and TMeOSMA in the absence and presence of 3 m NaCl. Scale bars=50 μm. (G) Absorbance at 600 nm (OD₆₀₀) of the mixture containing TMeOSMA and SBMA as a function of assembly time. (H) A diagram showing the range of droplet sizes in the mixtures containing SBMA and TMeOSMA. Error bars represent ±s.e.m. N=3.

FIG. 9 presents a schematic illustration (upper) and photographic (lower) images of the final products.

FIG. 10 provides linear rheological data of aqueous mixtures containing PCMA and TMeOSMA over time.

FIG. 11 presents phase contrast microscopy images of the SBMA/TMeOSMA mixture (ratio=5) varying total concentrations of precursor and NaCl. Scale bars=20 μm

FIG. 12A-D demonstrates hierarchical microstructures increase toughness and stretchability. (A) Schematic illustration of the photo-crosslinking process to fix the hierarchical microstructures from LLPS. (B) Representative photographic images of photo-crosslinked samples at 1.5-hour assembly time before and after applying axial tensile force. (C) Stress-strain curve of the mixture containing TMeOSMA and SBMA before (0 h) and after (3 h) hierarchical microstructure formation. (D) Toughness of the photo-crosslinked materials with different assembly times and droplet sizes. Error bars represent ±s.e.m. N=3.

FIG. 13A-B provides a stress-strain curve of the photocured materials containing SBMA/TMeOSMA (ratio=5, total concentration=5.7 m) at different assembly times in the (A) absence and (B) presence of 3 m NaCl.

FIG. 14A-B presents optical images of the mixtures containing SBMA and TMeOSMA (ratio=5, total concentration=5.7 m) photocured at (A) 0.5 h and (B) 1.5 h assembly times before and after applying axial tensile force.

FIG. 15A-H demonstrates tough and fast-acting underwater adhesives. (A) Schematic illustration of measuring underwater adhesion. (B) Lap shear adhesion strength values for glass substrate with different assembly times and droplet sizes. (C) Lap shear adhesion strength values when applied and cured under distilled water and seawater. (D) Photographic images of interfacial water containing CuSO4 (1 M) for visualization purposes upon applying adhesive precursors. (E) Lap shear adhesion strength values for porcine skin substrate. (F) Force-extension curve in the absence and presence of 1% NHSMA additive measured with porcine skin substrate. (G) Work of debonding of in the absence and presence of 1% NHSMA additive measured with porcine skin substrate. (H) Lap shear adhesion strength values in the absence and presence of 1% NHSMA additive measured with porcine skin substrate. Error bars represent ±s.e.m. N=3.

FIG. 16A-H provides force-strain curves of the adhered and cured glass substrates under distilled water. The adhesive mixture was prepared in the (A-D) absence and (E-H) presence of 3 m NaCl at different assembly times.

FIG. 17A-B presents optical images of the mixture of SBMA/TMeOSMA (ratio=5, total concentration=5.7 m) at (A) 0.5 h and (B) 1.5 h assembly times under water.

FIG. 18A-D provides force-strain curves of the adhered and cured glass substrates under seawater. The adhesive mixture was prepared in the absence of NaCl at a assembly time of 0.5h (A), 1.5 h (B), 2.5 h (C), and 3 h (D).

FIG. 19 shows adhesion strengths of the photocured mixture of SBMA/TMeOSMA deposited between two glass substrates under distilled water (light green) and seawater (gray). The mixture was prepared in the absence of NaCl with different assembly times.

FIG. 20A-C provides contact angle measurements of (A) water and (B, C) the adhesive mixture containing SBMA/TMeOSMA prepared in the (B) absence and (C) presence of 3 m NaCl on each substrate. One drop (2.5 μL) of the sample was deposited on glass, porcine skin, PMMA, and PP.

FIG. 21A-G provides force-strain curves of the adhered and cured PMMA substrates under distilled water. The adhesive mixture was prepared in the (A-C) absence and (D-G) presence of 3 m NaCl at different assembly times.

FIG. 22 shows the adhesion strengths of the photocured mixture of SBMA/TMeOSMA deposited between two PMMA substrates underwater.

FIG. 23A-H provides force-strain curves of the adhered and cured PP substrates under distilled water. The adhesive mixture was prepared in the (A-D) absence and (E-H) presence of 3 m NaCl at different assembly times.

FIG. 24 demonstrates the adhesion strengths of the photocured mixture of SBMA/TMeOSMA deposited between two PP substrates underwater.

FIG. 25A-D provides force-strain curves of the adhered and cured porcine skin substrates under distilled water. The adhesive mixture was prepared in the (A, B) absence and (C, D) presence of 3 m NaCl at different assembly times.

FIG. 26 shows the adhesion strengths of the photocured mixture of SBMA/TMeOSMA deposited between two porcine skin substrates underwater.

FIG. 27 provides force-strain curves of the adhered and cured porcine skin substrates with the mixture of SBMA/TMeOSMA containing 1% NHSMA under distilled water. 1% NHSMA was added to the mixture at 1.5 hours of assembly time.

FIG. 28 provides a stress-strain curve of the photocured materials before and after soaking in an aqueous solution of HCl (pH=3) for 3 h.

FIG. 29 shows a reaction set-up for glass adhesion underwater. Light was irradiated at a distance of 3.85 cm (THORLABS LED UV curing system, CS20K2, 365 nm).

### DETAILED DESCRIPTION

As used herein and in the appended claims, the singular forms “a,” “an,” and “the” include plural referents unless the context clearly dictates otherwise. Thus, for example, reference to “an underwater adhesive” includes a plurality of such adhesives and reference to “the component” includes reference to one or more components and equivalents thereof known to those skilled in the art, and so forth.

Also, the use of “or” means “and/or” unless stated otherwise. Similarly, “comprise,” “comprises,” “comprising”, “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

Other than in the operating examples, or where otherwise indicated, all numbers expressing quantities of ingredients or reaction conditions used herein should be understood as modified in all instances by the term “about.” The term “about” when used to describe the present invention, in connection with percentages means ±1%.

The term “a silyl-based compound” as used herein, refers to a compound that comprises one or more silicon based groups (e.g., silyl groups). “A silyl-based compound” may further comprise a moiety that can be polymerized under certain conditions, e.g., by addition of a radical initiator (e.g., a photoinitator). Examples of polymerizable moieties include, but are not limited to, methacrylate, acrylate, methacrylamide, and acrylamide.

The term “a zwitterion-based compound” as used herein, refers to a compound that has separate positively and negatively charged groups. “A zwitterion-based compound” may further comprise a moiety that can be polymerized under certain conditions, e.g., by addition of a radical initiator (e.g., a photoinitator). Examples of polymerizable moieties include, but are not limited to, methacrylate, acrylate, methacrylamide, and acrylamide.

Biology utilizes hierarchical structures under nonequilibrium conditions to program key functions, such as adhesion, transport, and protection. Importantly, phase separation of liquid precursors is a powerful mechanism to organize soft and amenable compartments into hierarchical structures in a range of length scales. This liquid-liquid phase separation (LLPS) process is driven by various noncovalent intermolecular forces and influenced by multivalent interactions in macromolecules. Typical thermodynamic minimum, especially in synthetic systems, is the macroscopic segregation of two phases to minimize the phase boundary. Small droplets growing from nuclei will coalesce into larger ones, and at the endpoint of LLPS, two distinct macroscopic layers emerge. Interfacial self-assembly to kinetically stabilize synthetic liquid droplets before macroscopic phase segregation has been shown with surfactants, macromolecules, inorganic colloids, and lipid membranes.

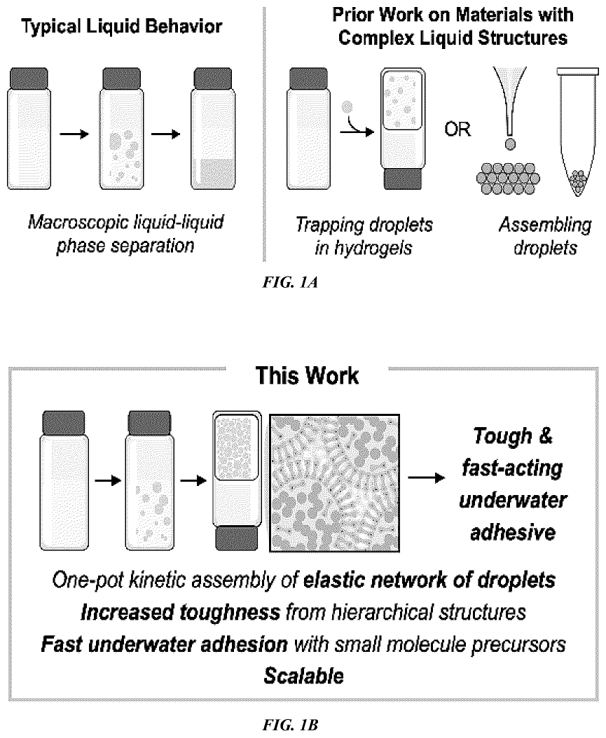

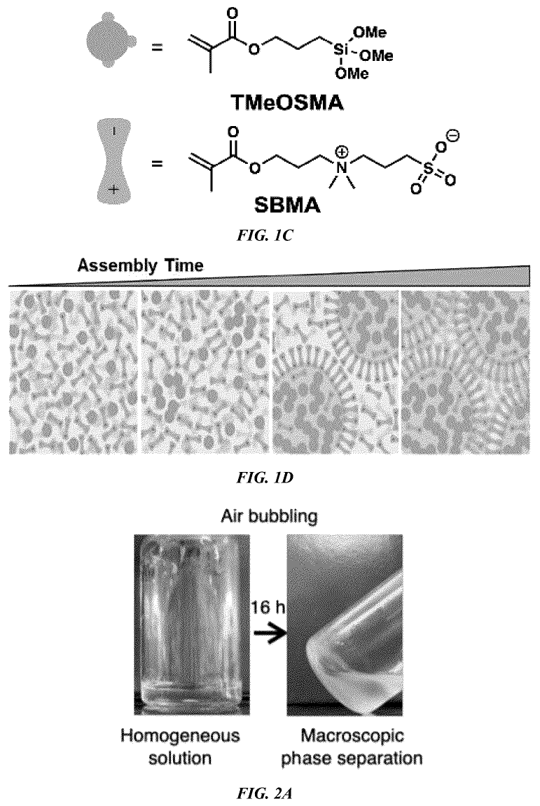

Efforts to construct macroscopic materials bearing kinetically trapped liquids have been made to recapitulate compartmentalized networks of liquid often found in biological structures, such as tissues (see FIG. 1A, right). For example, agarose hydrogels encasing droplets made of double-stranded DNA and dextran have been made by slow cooling the mixture from 60° C. to room temperature. Alternatively, assembling strategies for interface-stabilized droplets have also been demonstrated. Researchers have described a method to precisely control the placement and arrangement of lipid-stabilized liquid droplets using microfluidics and 3D droplet printers. Additional researchers have demonstrated spontaneous coalescence of polysaccharide-stabilized droplets by centrifugation. Finally, bicontinuous interfacially jammed emulsion gels (Bijel) were made with an emulsifying colloid at the interface between two liquids undergoing spinodal decomposition. Nonequilibrium bicontinuous morphologies are formed by the physical jamming of colloidal particles at the interface between the two liquids. Despite the progress in this area, a scalable method to produce an elastic network of liquids with desirable macroscopic functions is currently lacking.

In the studies presented herein, it was found the spontaneous formation of an elastic network of droplets via kinetically trapped self-assembly (see FIG. 1B). Distinct from previously reported systems of incorporating complex liquids within materials (see FIG. 1A), the viscoelastic material of the disclosure originates from one-pot mixtures of two small molecule liquid precursors, 3-(trimethoxysilyl)propyl methacrylate (TMeOSMA) and [2-(methacryloyloxy)ethyl]dimethyl-(3-sulfopropyl)ammonium hydroxide (sulfobetaine methacrylate, SBMA), undergoing hydrolysis, condensation, and LLPS at the same time (see FIG. 1C). Instead of macroscopic phase separation, a spontaneous formation of a viscoelastic material comprising compartmentalized liquid components was observed (see FIG. 1D). Subsequent polymerization on the methacrylate part of the network making up the viscoelastic material demonstrated that this type of hierarchical organization leads to increased toughness, stretchability, and adhesion. Further, it was shown that the viscoelastic material functions as photocurable and scalable underwater adhesives for a variety of substrates, including glass, porcine skin, and plastics.

Disclosed herein is a kinetically trapped self-assembly of a compartmentalized network of liquid droplets from small molecule precursors, a viscoelastic material, and its utility for tough and fast-acting underwater adhesive. In the studies presented herein, mixtures of two small molecules, TMeOSMA and SBMA, undergo concurrent hydrolysis, condensation, and LLPS to form a viscoelastic solid via interconnected liquid droplets. Zwitterionic SBMA molecule localizes on the surface of the droplet, and their molecularly interlocked structure drives the formation of the network. By photo-crosslinking the methacrylate part of the small molecule precursors, it was revealed that hierarchical microstructure increased the toughness, stretchability, and adhesion of the resulting viscoelastic materials. It was further demonstrated herein, the application of the viscoelastic material of the disclosure as a tough and fast-acting underwater adhesives for various substrates.

Functional, self-assembled hierarchical structures are one of the distinguishing characteristics of biological materials. The disclosure describes relatively simple processes to access complex hierarchical structures comprising liquid compartments. Distinct from prior studies in LLPS and protocell formation, the system disclosed herein to make the viscoelastic materials comprises multiple processes occurring at the same time: hydrolysis, condensation, LLPS, and self-organization of zwitterionic molecules on the droplet surface.

The simplistic nature of utilizing an aqueous one-pot assembly of scalable small molecule precursors is particularly meaningful for practical applications. Many reported synthetic underwater adhesives require elaborate macromolecular synthesis, impractical curing time, or the use of organic solvents in their formulation. The disclosure overcomes all the foregoing issues, and therefore broadens the scope of adhesives for a variety of applications, including underwater repairs or emergency wound care.

In a particular embodiment, the disclosure provides for a viscoelastic material having a solid or gel-like consistency, comprising: an elastic network of compartmentalized liquid droplets, wherein each liquid droplet has an exterior surface and inner compartment, wherein the inner compartment comprises a silyl-based compound and wherein the exterior surface comprises a zwitterion-based compound, wherein the electrostatic interactions between the compartmentalized liquid droplets interlock the compartmentalized liquid droplets together to form a viscoelastic material having a solid or gel-like consistency. In another embodiment, the elastic network of compartmentalized liquid droplets is a kinetic product. In yet another embodiment, the silyl-based compound forms interconnected silyl-based compounds via siloxane bonds. In a further embodiment, the silyl-based compound is selected from 3-(trimethoxysilyl)propyl methacrylate, 3-(trimethoxysilyl)methyl methacrylate, 3-(trimethoxysilyl)ethyl methacrylate, 3-(trimethoxysilyl)butyl methacrylate, 3-(dimethoxy(methyl)silyl)propyl methacrylate, 3-(dimethoxy(methyl)silyl)methyl methacrylate, 3-(dimethoxy(methyl)silyl)ethyl methacrylate, 3-(dimethoxy(methyl)silyl)butyl methacrylate, 3-(dimethyl(methoxy)silyl)propyl methacrylate, 3-(dimethyl(methoxy)silyl)methyl methacrylate, 3-(dimethyl(methoxy)silyl)ethyl methacrylate, 3-(dimethyl(methoxy)silyl)butyl methacrylate, (trimethylsilyl)methacrylate, 3-[Tris(trimethylsiloxy)silyl]propyl methacrylate, bis(trimethylsiloxy)methylsilyl methacrylate, triisopropylsilyl methacrylate, and tributylsilyl methacrylate. In yet a further embodiment, the silyl-based compound is 3-(trimethoxysilyl)propyl methacrylate (TMeOSMA). In another embodiment, the zwitterion-based compound is selected from 2-(methacryloyloxy)ethyl]dimethyl-(3-sulfopropyl)ammonium hydroxide, 2-(methacryloyloxy)ethyl]dimethyl-(3-sulfoethyl)ammonium hydroxide, 2-(methacryloyloxy)ethyl]dimethyl-(3-sulfobutyl)ammonium hydroxide, 3-3-[[2-(Methacryloyloxy)ethyl]dimethylammonio]propionate, 3-3-[[2-(Methacryloyloxy)ethyl]dimethylammonio]acetate, 3-3-[[2-(Methacryloyloxy)ethyl]dimethylammonio]butyrate, and 2-methacryloyloxyethyl phosphorylcholine. In yet another embodiment, the zwitterion-based polymerizable compound is 2-(methacryloyloxy)ethyl]dimethyl-(3-sulfopropyl)ammonium hydroxide (sulfobetaine methacrylate, SBMA). In a further embodiment, the ratio of the molality concentration of the zwitterion-based compound to the molality concentration of the silyl-based compound is 1:10, 1:5, 1:2, 1:1, 2:1, 3:1, 4:1, 5:1, 6:1, 7:1, 8:1, 10:1, 15:1, 20:1, 30:1, 40:1, or 50:1, or a range that includes or is in between any two of the foregoing ratios. In yet a further embodiment, the ratio of the molality concentration of the zwitterion-based compound to the molality concentration of the silyl-based compound is from 4:1 to 20:1. In another embodiment, the ratio of the molality concentration of the zwitterion-based compound to the molality concentration of the silyl-based compound is about 5:1. In yet another embodiment, the viscoelastic material further comprises a reactive monomer. In a further embodiment, the reactive monomer is methacrylic acid N-hydroxysuccinimide ester (NHSMA), methacrylic acid N-hydroxysuccinimide ester, N-(Hydroxymethyl)acrylamide acrylic acid, N-hydroxysuccinimide ester paraformaldehyde, glycidyl methacrlyate, and glycidyl acrylate. In yet a further embodiment, the viscoelastic material further comprises a photoinitiator. In another embodiment, the photoinitiator is selected from lithium phenyl-2,4,6-trimethylbenzoylphosphinate, sodium 4-[2-(4-morpholino)benzoyl-2-dimethylamino]butylbenzenesulphone (MBS), 2-hydroxy-1-[4-(2-hydroxyethoxy) phenyl]-2-methyl-1-propanone, BAPO-OLi, BAPO-ONa, and 2,2′-azobis[2-methyl-N-(2-hydroxyethyl) promionamide] (VA-086), isopropyl thioxanthone (ITX)-ethyl 4-(dimethylamino)benzoate, acetophenone, anisoin, anthraquinone, anthraquinone-2-sulfonic acid, (benzene) tricarbonylchromium, benzil, benzoin, benzoin ethyl ether, benzoin isobutyl ether, benzoin methyl ether, benzophenone, benzophenone/1-hydroxycyclohexyl phenyl ketone (50/50), 3,3′,4,4′-benzophenonetetracarboxylic dianhydride, 4-benzoylbiphenyl, 2-benzyl-2-(dimethylamino)-4′morpholinobutyrophenone, 4,4′-bis(diethylamino)benzophenone, camphorquinon, 2-chlorothioxanthen-9-one, (cumene)cyclopentadienyliron(II) hexafluorophosphate, dibenzosuberenone, 2,2-diethoxyacetophenone, 4,4′-dihydroxybenzophenone, 2,2-dimethoxy-2-phenylacetophenone, 4-(dimethylamino)benzophenone, 4,4′-dimethylbenzil, 2,5-dimethylbenzophenone, 3,4-dimethylbenzophenone, diphenyl(2,4,6-trimethylbenzoyl)phosphine oxide/2-hydroxy-2-methylpropiophenone (50/50), 4′-ethoxyacetophenone, 2-ethylanthraquinone, ferrocene, 3′-hydroxyacetophenone, 4′-hydroxyacetophenone, 3-hydroxybenzophenone, 4-hydroxybenzophenone, 1-hydroxycyclohexyl phenyl ketone, 2-hydroxy-2-methylpropiophenone, 2-methylbenzophenone, 3-methylbenzophenone, methybenzoylformate, 2-methyl-4′-(methylthio)-2-morpholinopropiophenone, phenanthrenequinone, 4′-phenoxyacetophenone, thioxanthen-9-one, triarylsulfonium hexafluoroantimonate salts and triarylsulfonium hexafluorophosphate salts. In yet another embodiment, the photoinitiator is a water soluble photoinitiator selected from lithium phenyl-2,4,6-trimethylbenzoylphosphinate (LAP), sodium 4-[2-(4-morpholino)benzoyl-2-dimethylamino]butylbenzenesulphone (MBS), 2-hydroxy-1-[4-(2-hydroxyethoxy) phenyl]-2-methyl-1-propanone, BAPO-OLi, BAPO-ONa, and 2,2′-azobis[2-methyl-N-(2-hydroxyethyl) promionamide] (VA-086). In a certain embodiment, the water soluble photoinitiator is LAP. In another embodiment, the viscoelastic material comprises LAP a final concentration from 1 mM, 2 mM, 3 mM, 4 mM, 5 mM, 6 mM, 7 mM, 8 mM, 9 mM, 10 mM, or a range that includes or is in between any two of the foregoing values including fractional increments thereof.

In a certain embodiment, the disclosure also provides an adhesive comprising the viscoelastic material of the disclosure. In a further embodiment, the adhesive is capable of adhering to a substrate that is exposed to an aqueous environment. In a further embodiment, the aqueous environment is water or saltwater.

In a particular embodiment, the disclosure further provides a bandage or dressing comprising the viscoelastic material disclosed herein, wherein the viscoelastic material is capable of adhering to biologically tissue. Examples of biological tissue include by are not limited to, skin tissue, hair tissue, nail tissue, corneal tissue, tongue tissue, oral cavity tissue, esophageal tissue, anal tissue, urethral tissue, vaginal tissue, urinary epithelial tissue, salivary gland tissue, mammary gland tissue, lacrimal gland tissue, sweat gland tissue, prostate gland tissue, bulbourethral gland tissue, Bartholin's gland tissue, uterine tissue, respiratory and gastrointestinal tract goblet cell tissue, gastric mucosal tissue, gastric gland tissue, pancreatic tissue, pulmonary tissue, pituitary gland tissue, thyroid gland tissue, parathyroid gland tissue, testicular tissue, ovarian tissue, respiratory gland tissue, gastrointestinal gland tissue, adrenal gland tissue, renal tissue, liver tissue, adipose tissue, duct cell tissue, gall bladder tissue, epidydimal tissue, vas deferens tissue, blood vessel tissue, lymph gland tissue, lymphatic duct tissue, synovial tissue, serosal tissue, squamous tissue, cochlear tissue, choroid plexus tissue, ependymal tissue, dural tissue, pia-arachnoid tissue, sclera tissue, retinal tissue, iris tissue, ciliary tissue, dental tissue, otic tissue, ligament tissue, tendon tissue, elastic cartilage tissue, fibrocartilage tissue, hyaline cartilage tissue, bone marrow tissue, intervertebral disc tissue, compact bone tissue, cancellous bone tissue, skeletal muscle tissue, cardiac muscle tissue, smooth muscle tissue, cardiac valve tissue, pericardial tissue, pleural tissue, peritoneal tissue, blood cell tissue, neuronal tissue, glial tissue, sensory transducer cell tissue, pain sensitive tissue, autonomic neuron tissue, peripheral nervous system tissue, cranial nerve tissue, ocular lens tissue, germ cell tissue, thymus tissue, placental tissue, fetal membrane tissue, umbilical tissue, stem cell tissue, mesodermal tissue, ectodermal tissue, endodermal tissue, autologous tissue, allograft tissue or a combination thereof. In a particular embodiment, the biological tissue is skin tissue.

In a particular embodiment, the disclosure provides a process of manufacturing a viscoelastic material disclosed herein, comprising: forming a mixture by combining a silyl-based compound and an acid, to an aqueous solution comprising a zwitterion-based compound; vigorously introducing a gas into the mixture to form a homogenous mixture; optionally, adding a photoinitator and/or a reactive monomer to the homogenous mixture; solidifying the homogenous mixture into a viscoelastic material having a solid or gel-like consistency, wherein the homogenous mixture spontaneously forms the viscoelastic material over a period of time. In a further embodiment, the mixture further comprises a salt. In yet a further embodiment, the salt is selected from sodium chloride, potassium chloride, and calcium chloride. In yet a further embodiment, the mixture comprises the salt at a molality of 1 m, 2 m, 3 m, 4 m, 5 m, 6 m, 7 m, 8 m, 9 m, 10 m, or a range that includes or is in between any two of the foregoing values. In a certain embodiment, the mixture has a pH from 1 to 6. In another embodiment, the mixture has a pH of 0, 1, 2, 3, 4, 5, 6, or a range that includes or is in between any two of the foregoing values. In a further embodiment, the silyl-based compound and/or the zwitterion-based compound comprises a polymerizable moiety selected from methacrylate, acrylate, methacrylamide, and acrylamide. IN another embodiment, the silyl-based compound is selected from 3-(trimethoxysilyl)propyl methacrylate, 3-(trimethoxysilyl)methyl methacrylate, 3-(trimethoxysilyl)ethyl methacrylate, 3-(trimethoxysilyl)butyl methacrylate, 3-(dimethoxy(methyl)silyl)propyl methacrylate, 3-(dimethoxy(methyl)silyl)methyl methacrylate, 3-(dimethoxy(methyl)silyl)ethyl methacrylate, 3-(dimethoxy(methyl)silyl)butyl methacrylate, 3-(dimethyl(methoxy)silyl)propyl methacrylate, 3-(dimethyl(methoxy)silyl)methyl methacrylate, 3-(dimethyl(methoxy)silyl)ethyl methacrylate, 3-(dimethyl(methoxy)silyl)butyl methacrylate, (trimethylsilyl)methacrylate, 3-[Tris(trimethylsiloxy)silyl]propyl methacrylate, bis(trimethylsiloxy)methylsilyl methacrylate, triisopropylsilyl methacrylate, and/or tributylsilyl methacrylate. In a certain embodiment, the silyl methacrylate-based compound is 3-(trimethoxysilyl)propyl methacrylate. In a further embodiment, the zwitterion methacrylate-based compound is selected from 2-(methacryloyloxy)ethyl]dimethyl-(3-sulfopropyl)ammonium hydroxide, 2-(methacryloyloxy)ethyl]dimethyl-(3-sulfoethyl)ammonium hydroxide, 2-(methacryloyloxy)ethyl]dimethyl-(3-sulfobutyl)ammonium hydroxide, 3-3-[[2-(Methacryloyloxy)ethyl]dimethylammonio]propionate, 3-3-[[2-(Methacryloyloxy)ethyl]dimethylammonio]acetate, 3-3-[[2-(Methacryloyloxy)ethyl]dimethylammonio]butyrate, and 2-methacryloyloxyethyl phosphorylcholine. In yet a further embodiment, the zwitterion methacrylate-based compound is 2-(methacryloyloxy)ethyl]dimethyl-(3-sulfopropyl)ammonium hydroxide. In a certain embodiment, the acid is hydrochloric acid or sulfuric acid. In yet another embodiment, the gas that is bubbled through the mixture is selected from air, nitrogen gas, helium gas, argon gas, or carbon dioxide. In certain embodiment, the gas that is bubbled through the acidic aqueous solution is nitrogen gas. In a further embodiment, the homogenous mixture comprises the reactive monomer. In yet a further embodiment, the reactive monomer is methyacrylic acid N-hydroxysuccinimide ester, methacrylic acid N-hydroxysuccinimide ester, N-(Hydroxymethyl)acrylamide acrylic acid, N-hydroxysuccinimide ester paraformaldehyde, glycidyl methacrlyate, and glycidyl acrylate. In another embodiment, the homogenous mixture comprises the photoinitator. In a further embodiment, the photoinitator is selected from lithium phenyl-2,4,6-trimethylbenzoylphosphinate, sodium 4-[2-(4-morpholino)benzoyl-2-dimethylamino]butylbenzenesulphone (MBS), 2-hydroxy-1-[4-(2-hydroxyethoxy) phenyl]-2-methyl-1-propanone, BAPO-OLi, BAPO-ONa, and 2,2′-azobis[2-methyl-N-(2-hydroxyethyl) promionamide] (VA-086), isopropyl thioxanthone (ITX)-ethyl 4-(dimethylamino)benzoate, acetophenone, anisoin, anthraquinone, anthraquinone-2-sulfonic acid, (benzene) tricarbonylchromium, benzil, benzoin, benzoin ethyl ether, benzoin isobutyl ether, benzoin methyl ether, benzophenone, benzophenone/1-hydroxycyclohexyl phenyl ketone (50/50), 3,3′,4,4′-benzophenonetetracarboxylic dianhydride, 4-benzoylbiphenyl, 2-benzyl-2-(dimethylamino)-4′morpholinobutyrophenone, 4,4′-bis(diethylamino)benzophenone, camphorquinon, 2-chlorothioxanthen-9-one, (cumene)cyclopentadienyliron(II) hexafluorophosphate, dibenzosuberenone, 2,2-diethoxyacetophenone, 4,4′-dihydroxybenzophenone, 2,2-dimethoxy-2-phenylacetophenone, 4-(dimethylamino)benzophenone, 4,4′-dimethylbenzil, 2,5-dimethylbenzophenone, 3,4-dimethylbenzophenone, diphenyl(2,4,6-trimethylbenzoyl)phosphine oxide/2-hydroxy-2-methylpropiophenone (50/50), 4′-ethoxyacetophenone, 2-ethylanthraquinone, ferrocene, 3′-hydroxyacetophenone, 4′-hydroxyacetophenone, 3-hydroxybenzophenone, 4-hydroxybenzophenone, 1-hydroxycyclohexyl phenyl ketone, 2-hydroxy-2-methylpropiophenone, 2-methylbenzophenone, 3-methylbenzophenone, methybenzoylformate, 2-methyl-4′-(methylthio)-2-morpholinopropiophenone, phenanthrenequinone, 4′-phenoxyacetophenone, thioxanthen-9-one, triarylsulfonium hexafluoroantimonate salts and triarylsulfonium hexafluorophosphate salts. In another embodiment, the photoinitiator is a water soluble photoinitiator selected from lithium phenyl-2,4,6-trimethylbenzoylphosphinate (LAP), sodium 4-[2-(4-morpholino)benzoyl-2-dimethylamino]butylbenzenesulphone (MBS), 2-hydroxy-1-[4-(2-hydroxyethoxy) phenyl]-2-methyl-1-propanone, BAPO-OLi, BAPO-ONa, and 2,2′-azobis[2-methyl-N-(2-hydroxyethyl) promionamide] (VA-086). In a certain embodiment, the photoinitiator is LAP. In another embodiment, the process further comprises the step of: curing or fixing the viscoelastic material by exposing the viscoelastic material to light having a wavelength in the ultraviolet-visible spectral range.

In a particular embodiment, the disclosure provides a process of manufacturing a viscoelastic material disclosed herein, comprising: forming a mixture by combining a silyl-based compound and an acid, to an aqueous solution comprising a zwitterion-based compound; vigorously introducing a gas into the mixture to form a homogenous mixture; optionally, adding a photoinitator and/or a reactive monomer to the homogenous mixture; solidifying the homogenous mixture into a viscoelastic material having a solid or gel-like consistency, wherein the homogenous mixture spontaneously forms the viscoelastic material over a period of time. In another embodiment, the silyl-based compound is 3-(trimethoxysilyl)propyl methacrylate (TMeOSMA); the zwitterion-based compound is 2-(methacryloyloxy)ethyl]dimethyl-(3-sulfopropyl)ammonium hydroxide (SBMA); the pH of the mixture is from 1 to 4; and/or the gas is nitrogen gas. In yet another embodiment, the homogenous mixture comprises the photoiniator, or comprises the photoiniator and the reactive monomer, and the method further comprises the step of: curing or fixing the viscoelastic material by exposing the viscoelastic material to light having a wavelength in the ultraviolet-visible spectral range, wherein the photoiniator is lithium phenyl-2,4,6-trimethylbenzoylphosphinate (LAP) and the reactive monomer is methacrylic acid N-hydroxysuccinimide ester (NHSMA).

In a particular embodiment, the disclosure also provides for a viscoelastic material made by a process disclosed herein.

In a certain embodiment, the disclosure also provides for an adhesive comprising a viscoelastic material made by a process as substantially disclosed herein. In a further embodiment, the adhesive is capable of adhering to a substrate that is exposed to an aqueous environment. In yet a further embodiment, the aqueous environment comprises water that comprises from 0 ppt up to 50 parts per thousand of dissolved salts. In another embodiment, the aqueous environment is fresh water, brackish water, saline, seawater, or brine.

In a particular embodiment, the disclosure provides for a bandage or dressing comprising a viscoelastic material made by a process disclosed herein, wherein the viscoelastic material is capable of adhering to biological tissue.

The compositions and processes of the disclosure overcome major limitations of other polymer-based adhesives by using two scalable small molecule precursors instead of multi-step synthesis of designer polymers. The processes disclosed herein can be scaled up to a commercial scale and the resulting viscoelastic materials can find use in many industries. For example, the viscoelastic materials can be used in medical (e.g., adhesive for dressing and bandages) and watercraft industries (e.g., underwater adhesive). For underwater adhesive applications, a photoinitator can be added to sticky viscoelastic material, and the resulting adhesive can be applied to surfaces underwater, and then fixed by photocuring the adhesive in mere minutes. Due to the viscoelastic material disclosed herein having an elastic network of compartmentalized liquid droplets, surface adhesion, stable deposition underwater, and toughness can be all realized. Accordingly, the disclosure provides a platform for robust adhesives for rapid underwater repair or emergency wound care.

For use in applications described herein, articles of manufacture or kits are also described herein. Such articles of manufacture or kits can comprise a carrier, package, or container that is compartmentalized to receive one or more containers, each of the container(s) comprising one or more of the components to make a viscoelastic material disclosed herein, or the viscoelastic materials themselves, to be used in a method described herein. Suitable containers include, for example, tanks, carboys, drums, bottles, vials, syringes, and test tubes. The containers can be formed from a variety of materials such as glass or plastic. For example, the container(s) can comprise one or more of the components to make a viscoelastic material disclosed herein, or the viscoelastic materials themselves. Such articles of manufacture or kits can optionally comprise an identifying description or label or instructions relating to its use in a method described herein.

An article of manufacture or kit can comprise one or more additional containers, each with one or more of various materials (such as reagents, optionally in concentrated form, and/or devices) desirable from a commercial and user standpoint for use of a viscoelastic material described herein. Non-limiting examples of such materials include, but are not limited to, buffers, diluents, filters and/or labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific application. The label can also indicate directions for use of the contents, such as in the methods described herein.

The disclosure further provides that the methods and compositions described herein can be further defined by the following aspects (aspects 1 to 52):

1. A viscoelastic material having a solid or gel-like consistency, comprising: an elastic network of compartmentalized liquid droplets, wherein each liquid droplet has an exterior surface and inner compartment, wherein the inner compartment comprises a silyl-based compound and wherein the exterior surface comprises a zwitterion-based compound, wherein the electrostatic interactions between the compartmentalized liquid droplets interlock the compartmentalized liquid droplets together to form a viscoelastic material having a solid or gel-like consistency.

2. The viscoelastic material of aspect 1, wherein the elastic network of compartmentalized liquid droplets is a kinetic product.

3. The viscoelastic material of aspect 1 or aspect 2, wherein the silyl-based compound forms interconnected silyl-based compounds via siloxane bonds.

4. The viscoelastic material of any one of aspects 1 to 3, wherein the silyl-based compound is selected from 3-(trimethoxysilyl)propyl methacrylate, 3-(trimethoxysilyl)methyl methacrylate, 3-(trimethoxysilyl)ethyl methacrylate, 3-(trimethoxysilyl)butyl methacrylate, 3-(dimethoxy(methyl)silyl)propyl methacrylate, 3-(dimethoxy(methyl)silyl)methyl methacrylate, 3-(dimethoxy(methyl)silyl)ethyl methacrylate, 3-(dimethoxy(methyl)silyl)butyl methacrylate, 3-(dimethyl(methoxy)silyl)propyl methacrylate, 3-(dimethyl(methoxy)silyl)methyl methacrylate, 3-(dimethyl(methoxy)silyl)ethyl methacrylate, 3-(dimethyl(methoxy)silyl)butyl methacrylate, (trimethylsilyl)methacrylate, 3-[Tris(trimethylsiloxy)silyl]propyl methacrylate, bis(trimethylsiloxy)methylsilyl methacrylate, triisopropylsilyl methacrylate, and tributylsilyl methacrylate.

5. The viscoelastic material of any one of aspects 1 to 4, wherein the silyl-based compound is 3-(trimethoxysilyl)propyl methacrylate (TMeOSMA).

6. The viscoelastic material of any one of aspects 1 to 5, wherein the zwitterion-based compound is selected from 2-(methacryloyloxy)ethyl]dimethyl-(3-sulfopropyl)ammonium hydroxide, 2-(methacryloyloxy)ethyl]dimethyl-(3-sulfoethyl)ammonium hydroxide, 2-(methacryloyloxy)ethyl]dimethyl-(3-sulfobutyl)ammonium hydroxide, 3-3-[[2-(Methacryloyloxy)ethyl]dimethylammonio]propionate, 3-3-[[2-(Methacryloyloxy)ethyl]dimethylammonio]acetate, 3-3-[[2-(Methacryloyloxy)ethyl]dimethylammonio]butyrate, and 2-methacryloyloxyethyl phosphorylcholine.

7. The viscoelastic material of any one of aspects 1 to 6, wherein the zwitterion-based polymerizable compound is 2-(methacryloyloxy)ethyl]dimethyl-(3-sulfopropyl)ammonium hydroxide (SBMA).

8. The viscoelastic material of any one of aspects 1 to 7, wherein the ratio of the molality concentration of the zwitterion-based compound to the molality concentration of the silyl-based compound is from 2:1 to 50:1.

9. The viscoelastic material of any one of aspects 1 to 8, wherein the ratio of the molality concentration of the zwitterion-based compound to the molality concentration of the silyl-based compound is from 4:1 to 20:1.

10. The viscoelastic material of any one of aspects 1 to 9, wherein the ratio of the molality concentration of the zwitterion-based compound to the molality concentration of the silyl-based compound is about 5:1.

11. The viscoelastic material of any one of aspects 1 to 10, wherein the viscoelastic material further comprises a reactive monomer.

12. The viscoelastic material of aspect 11, wherein the reactive monomer is methacrylic acid N-hydroxysuccinimide ester (NHSMA), methacrylic acid N-hydroxysuccinimide ester, N-(Hydroxymethyl)acrylamide acrylic acid, N-hydroxysuccinimide ester paraformaldehyde, glycidyl methacrlyate, and glycidyl acrylate.

13. The viscoelastic material of any one of aspects 1 to 12, wherein the viscoelastic material further comprises a photoinitiator.

14. The viscoelastic material of aspect 13, wherein the photoinitiator is selected from lithium phenyl-2,4,6-trimethylbenzoylphosphinate, sodium 4-[2-(4-morpholino)benzoyl-2-dimethylamino]butylbenzenesulphone (MBS), 2-hydroxy-1-[4-(2-hydroxyethoxy) phenyl]-2-methyl-1-propanone, BAPO-OLi, BAPO-ONa, and 2,2′-azobis[2-methyl-N-(2-hydroxyethyl) promionamide] (VA-086), isopropyl thioxanthone (ITX)-ethyl 4-(dimethylamino)benzoate, acetophenone, anisoin, anthraquinone, anthraquinone-2-sulfonic acid, (benzene) tricarbonylchromium, benzil, benzoin, benzoin ethyl ether, benzoin isobutyl ether, benzoin methyl ether, benzophenone, benzophenone/1-hydroxycyclohexyl phenyl ketone (50/50), 3,3′,4,4′-benzophenonetetracarboxylic dianhydride, 4-benzoylbiphenyl, 2-benzyl-2-(dimethylamino)-4′morpholinobutyrophenone, 4,4′-bis(diethylamino)benzophenone, camphorquinon, 2-chlorothioxanthen-9-one, (cumene)cyclopentadienyliron(II) hexafluorophosphate, dibenzosuberenone, 2,2-diethoxyacetophenone, 4,4′-dihydroxybenzophenone, 2,2-dimethoxy-2-phenylacetophenone, 4-(dimethylamino)benzophenone, 4,4′-dimethylbenzil, 2,5-dimethylbenzophenone, 3,4-dimethylbenzophenone, diphenyl(2,4,6-trimethylbenzoyl)phosphine oxide/2-hydroxy-2-methylpropiophenone (50/50), 4′-ethoxyacetophenone, 2-ethylanthraquinone, ferrocene, 3′-hydroxyacetophenone, 4′-hydroxyacetophenone, 3-hydroxybenzophenone, 4-hydroxybenzophenone, 1-hydroxycyclohexyl phenyl ketone, 2-hydroxy-2-methylpropiophenone, 2-methylbenzophenone, 3-methylbenzophenone, methybenzoylformate, 2-methyl-4′-(methylthio)-2-morpholinopropiophenone, phenanthrenequinone, 4′-phenoxyacetophenone, thioxanthen-9-one, triarylsulfonium hexafluoroantimonate salts and triarylsulfonium hexafluorophosphate salts.

15. The viscoelastic material of aspect 13 or aspect 14, wherein the photoinitiator is a water soluble photoinitiator selected from lithium phenyl-2,4,6-trimethylbenzoylphosphinate (LAP), sodium 4-[2-(4-morpholino)benzoyl-2-dimethylamino]butylbenzenesulphone (MBS), 2-hydroxy-1-[4-(2-hydroxyethoxy) phenyl]-2-methyl-1-propanone, BAPO-OLi, BAPO-ONa, and 2,2′-azobis[2-methyl-N-(2-hydroxyethyl) promionamide] (VA-086).

16. The viscoelastic material of aspect 15, wherein the water soluble photoinitiator is LAP.

17. The viscoelastic material of aspect 16, wherein the viscoelastic material comprises LAP a final concentration from 2 mM to 4 mM.

18. An adhesive comprising the viscoelastic material of any one of aspects 1 to 17, wherein the adhesive is capable of adhering to a substrate that is exposed to an aqueous environment.

19. The adhesive of aspect 18, wherein the aqueous environment comprises water that has from 0 ppt up to 50 parts per thousand of dissolved salts.

20. The adhesive of aspect 18, wherein the aqueous environment is fresh water, brackish water, saline, seawater, or brine.

21. A bandage, dressing, or medical implant comprising the viscoelastic material of any one of aspects 1 to 17, wherein the viscoelastic material is capable of adhering to biological tissue.

22. The bandage, dressing, or medical implant of aspect 21, wherein the biological tissue is selected from skin tissue, hair tissue, nail tissue, corneal tissue, tongue tissue, oral cavity tissue, esophageal tissue, anal tissue, urethral tissue, vaginal tissue, urinary epithelial tissue, salivary gland tissue, mammary gland tissue, lacrimal gland tissue, sweat gland tissue, prostate gland tissue, bulbourethral gland tissue, Bartholin's gland tissue, uterine tissue, respiratory and gastrointestinal tract goblet cell tissue, gastric mucosal tissue, gastric gland tissue, pancreatic tissue, pulmonary tissue, pituitary gland tissue, thyroid gland tissue, parathyroid gland tissue, testicular tissue, ovarian tissue, respiratory gland tissue, gastrointestinal gland tissue, adrenal gland tissue, renal tissue, liver tissue, adipose tissue, duct cell tissue, gall bladder tissue, epidydimal tissue, vas deferens tissue, blood vessel tissue, lymph gland tissue, lymphatic duct tissue, synovial tissue, serosal tissue, squamous tissue, cochlear tissue, choroid plexus tissue, ependymal tissue, dural tissue, pia-arachnoid tissue, sclera tissue, retinal tissue, iris tissue, ciliary tissue, dental tissue, otic tissue, ligament tissue, tendon tissue, elastic cartilage tissue, fibrocartilage tissue, hyaline cartilage tissue, bone marrow tissue, intervertebral disc tissue, compact bone tissue, cancellous bone tissue, skeletal muscle tissue, cardiac muscle tissue, smooth muscle tissue, cardiac valve tissue, pericardial tissue, pleural tissue, peritoneal tissue, blood cell tissue, neuronal tissue, glial tissue, sensory transducer cell tissue, pain sensitive tissue, autonomic neuron tissue, peripheral nervous system tissue, cranial nerve tissue, ocular lens tissue, germ cell tissue, thymus tissue, placental tissue, fetal membrane tissue, umbilical tissue, stem cell tissue, mesodermal tissue, ectodermal tissue, endodermal tissue, autologous tissue, allograft tissue or a combination thereof.

23. The bandage, dressing, or medical implant of aspect 22, wherein the biological tissue is skin tissue.

24. A process of manufacturing the viscoelastic material of any one of aspects 1 to 17, comprising:
forming a mixture by combining a silyl-based compound and an acid, to an aqueous solution comprising a zwitterion-based compound;
vigorously introducing a gas into the mixture to form a homogenous mixture; optionally, adding a photoinitator and/or a reactive monomer to the homogenous mixture;
solidifying the homogenous mixture into a viscoelastic material having a solid or gel-like consistency, wherein the homogenous mixture spontaneously forms the viscoelastic material over a period of time.

25. The process of aspect 24, wherein the aqueous solution further comprises a salt.

26. The process of aspect 25, wherein the salt is selected from sodium chloride, potassium chloride, and calcium chloride.

27. The process of aspect 25 or aspect 26, wherein the aqueous solution comprises the salt at a molality from 0 m to 10 m.

28. The process of any one of aspects 24 to 27, wherein the mixture has a pH from 1 to 6.

29. The process of any one of aspects 24 to 28, wherein the mixture has a pH from 1 to 4.

30. The process of any one of aspects 24 to 29, wherein the silyl-based compound and/or the zwitterion-based compound comprises a polymerizable moiety selected from methacrylate, acrylate, methacrylamide, and acrylamide.

31. The process of any one of aspects 23 to 30, wherein the silyl-based compound is selected from 3-(trimethoxysilyl)propyl methacrylate, 3-(trimethoxysilyl)methyl methacrylate, 3-(trimethoxysilyl)ethyl methacrylate, 3-(trimethoxysilyl)butyl methacrylate, 3-(dimethoxy(methyl)silyl)propyl methacrylate, 3-(dimethoxy(methyl)silyl)methyl methacrylate, 3-(dimethoxy(methyl)silyl)ethyl methacrylate, 3-(dimethoxy(methyl)silyl)butyl methacrylate, 3-(dimethyl(methoxy)silyl)propyl methacrylate, 3-(dimethyl(methoxy)silyl)methyl methacrylate, 3-(dimethyl(methoxy)silyl)ethyl methacrylate, 3-(dimethyl(methoxy)silyl)butyl methacrylate, (trimethylsilyl)methacrylate, 3-[Tris(trimethylsiloxy)silyl]propyl methacrylate, bis(trimethylsiloxy)methylsilyl methacrylate, triisopropylsilyl methacrylate, and/or tributylsilyl methacrylate.

32. The process of any one of aspects 24 to 31, wherein the silyl methacrylate-based compound is 3-(trimethoxysilyl)propyl methacrylate.

33. The process of any one of aspects 24 to 32, wherein the zwitterion methacrylate-based polymerizable compound is selected from 2-(methacryloyloxy)ethyl]dimethyl-(3-sulfopropyl)ammonium hydroxide, 2-(methacryloyloxy)ethyl]dimethyl-(3-sulfoethyl)ammonium hydroxide, 2-(methacryloyloxy)ethyl]dimethyl-(3-sulfobutyl)ammonium hydroxide, 3-3-[[2-(Methacryloyloxy)ethyl]dimethylammonio]propionate, 3-3-[[2-(Methacryloyloxy)ethyl]dimethylammonio]acetate, 3-3-[[2-(Methacryloyloxy)ethyl]dimethylammonio]butyrate, and 2-methacryloyloxyethyl phosphorylcholine.

34. The process of any one of aspects 24 to 33, wherein the zwitterion methacrylate-based compound is 2-(methacryloyloxy)ethyl]dimethyl-(3-sulfopropyl)ammonium hydroxide.

35. The process of any one of aspects 24 to 34, wherein the acid is hydrochloric acid or sulfuric acid.

36. The process of any one of aspects 24 to 35, wherein the gas that is bubbled through mixture is selected from air, nitrogen gas, helium gas, argon gas, or carbon dioxide.

37. The process of aspect 36, wherein the gas that is bubbled through the mixture is nitrogen gas.

38. The process of any one of aspects 24 to 37, wherein the homogenous mixture comprises the reactive monomer.

39. The process of aspect 38, wherein the reactive monomer is selected from methyacrylic acid N-hydroxysuccinimide ester, methacrylic acid N-hydroxysuccinimide ester, N-(Hydroxymethyl)acrylamide acrylic acid, N-hydroxysuccinimide ester paraformaldehyde, glycidyl methacrlyate, and glycidyl acrylate.

40. The process of any one of aspects 24 to 39, wherein the homogenous mixture comprises the photoinitator.

41. The process of aspect 40, wherein the photoinitator is selected from lithium phenyl-2,4,6-trimethylbenzoylphosphinate, sodium 4-[2-(4-morpholino)benzoyl-2-dimethylamino]butylbenzenesulphone (MBS), 2-hydroxy-1-[4-(2-hydroxyethoxy) phenyl]-2-methyl-1-propanone, BAPO-OLi, BAPO-ONa, and 2,2′-azobis[2-methyl-N-(2-hydroxyethyl) promionamide] (VA-086), isopropyl thioxanthone (ITX)-ethyl 4-(dimethylamino)benzoate, acetophenone, anisoin, anthraquinone, anthraquinone-2-sulfonic acid, (benzene) tricarbonylchromium, benzil, benzoin, benzoin ethyl ether, benzoin isobutyl ether, benzoin methyl ether, benzophenone, benzophenone/1-hydroxycyclohexyl phenyl ketone (50/50), 3,3′,4,4′-benzophenonetetracarboxylic dianhydride, 4-benzoylbiphenyl, 2-benzyl-2-(dimethylamino)-4′morpholinobutyrophenone, 4,4′-bis(diethylamino)benzophenone, camphorquinon, 2-chlorothioxanthen-9-one, (cumene)cyclopentadienyliron(II) hexafluorophosphate, dibenzosuberenone, 2,2-diethoxyacetophenone, 4,4′-dihydroxybenzophenone, 2,2-dimethoxy-2-phenylacetophenone, 4-(dimethylamino)benzophenone, 4,4′-dimethylbenzil, 2,5-dimethylbenzophenone, 3,4-dimethylbenzophenone, diphenyl(2,4,6-trimethylbenzoyl)phosphine oxide/2-hydroxy-2-methylpropiophenone (50/50), 4′-ethoxyacetophenone, 2-ethylanthraquinone, ferrocene, 3′-hydroxyacetophenone, 4′-hydroxyacetophenone, 3-hydroxybenzophenone, 4-hydroxybenzophenone, 1-hydroxycyclohexyl phenyl ketone, 2-hydroxy-2-methylpropiophenone, 2-methylbenzophenone, 3-methylbenzophenone, methybenzoylformate, 2-methyl-4′-(methylthio)-2-morpholinopropiophenone, phenanthrenequinone, 4′-phenoxyacetophenone, thioxanthen-9-one, triarylsulfonium hexafluoroantimonate salts and triarylsulfonium hexafluorophosphate salts.

42. The process of aspect 40 or aspect 41, wherein the photoinitiator is a water soluble photoinitiator selected from lithium phenyl-2,4,6-trimethylbenzoylphosphinate (LAP), sodium 4-[2-(4-morpholino)benzoyl-2-dimethylamino]butylbenzenesulphone (MBS), 2-hydroxy-1-[4-(2-hydroxyethoxy) phenyl]-2-methyl-1-propanone, BAPO-OLi, BAPO-ONa, and 2,2′-azobis[2-methyl-N-(2-hydroxyethyl) promionamide] (VA-086).

43. The process of any one of aspects 40 to 42, wherein the photoinitiator is LAP.

44. The process of any one of aspects 40 to 43, wherein the process further comprises the step of:
curing or fixing the viscoelastic material by exposing the viscoelastic material to light having a wavelength in the ultraviolet-visible spectral range.

45. A viscoelastic material made by the process of any one of aspect 24 to 44.

46. An adhesive comprising the viscoelastic material of aspect 45.

47. An adhesive of aspect 46, wherein the adhesive is capable of adhering to a substrate that is exposed to an aqueous environment.

48. The adhesive of aspect 47, wherein the aqueous environment comprises water that comprises from 0 ppt up to 50 parts per thousand of dissolved salts.

49. The adhesive of aspect 47, wherein the aqueous environment is fresh water, brackish water, saline, seawater, or brine.

50. A bandage, dressing, or medical implant comprising the adhesive of any one of aspects 46 to 49, wherein the adhesive is capable of adhering to biological tissue.

51. The bandage, dressing, or medical implant of aspect 50, wherein the biological tissue is selected from skin tissue, hair tissue, nail tissue, corneal tissue, tongue tissue, oral cavity tissue, esophageal tissue, anal tissue, urethral tissue, vaginal tissue, urinary epithelial tissue, salivary gland tissue, mammary gland tissue, lacrimal gland tissue, sweat gland tissue, prostate gland tissue, bulbourethral gland tissue, Bartholin's gland tissue, uterine tissue, respiratory and gastrointestinal tract goblet cell tissue, gastric mucosal tissue, gastric gland tissue, pancreatic tissue, pulmonary tissue, pituitary gland tissue, thyroid gland tissue, parathyroid gland tissue, testicular tissue, ovarian tissue, respiratory gland tissue, gastrointestinal gland tissue, adrenal gland tissue, renal tissue, liver tissue, adipose tissue, duct cell tissue, gall bladder tissue, epidydimal tissue, vas deferens tissue, blood vessel tissue, lymph gland tissue, lymphatic duct tissue, synovial tissue, serosal tissue, squamous tissue, cochlear tissue, choroid plexus tissue, ependymal tissue, dural tissue, pia-arachnoid tissue, sclera tissue, retinal tissue, iris tissue, ciliary tissue, dental tissue, otic tissue, ligament tissue, tendon tissue, elastic cartilage tissue, fibrocartilage tissue, hyaline cartilage tissue, bone marrow tissue, intervertebral disc tissue, compact bone tissue, cancellous bone tissue, skeletal muscle tissue, cardiac muscle tissue, smooth muscle tissue, cardiac valve tissue, pericardial tissue, pleural tissue, peritoneal tissue, blood cell tissue, neuronal tissue, glial tissue, sensory transducer cell tissue, pain sensitive tissue, autonomic neuron tissue, peripheral nervous system tissue, cranial nerve tissue, ocular lens tissue, germ cell tissue, thymus tissue, placental tissue, fetal membrane tissue, umbilical tissue, stem cell tissue, mesodermal tissue, ectodermal tissue, endodermal tissue, autologous tissue, allograft tissue or a combination thereof.

52. The bandage, dressing, or medical implant of aspect 51, wherein the biological tissue is skin tissue.

The following examples are intended to illustrate but not limit the disclosure. While they are typical of those that might be used, other procedures known to those skilled in the art may alternatively be used.

### Examples

The invention is illustrated in the following examples, which are provided by way of illustration and are not intended to be limiting.

Materials: Reagents and solvents were used as received from commercial sources without further purification. Trimethoxylsilyl methacrylate (TMeOSMA), [2-(methacryloyloxy)ethyl]dimethyl-(3-sulfopropyl)ammonium hydroxide (sulfobetainemethacrylate, SBMA), 2-methacryloyloxyethyl phosphorylcholine (phosphorylcholine methacrylate, PCMA), and lithium phenyl-2,4,6-trimethylbenzoylphosphinate (LAP) were purchased from Sigma-Aldrich (MO, USA). 3-3-[[2-(Methacryloyloxy)ethyl]dimethylammonio]propionate (carboxybetaine methacrylate, CBMA)was purchased from TCI (USA). SBMA was stored in a desiccator, and CBMA and PBMA were kept at 4° C. filled with N₂ to avoid water accumulation. FAM-SH was purchased from BioActs (South Korea). Glass substrate was purchased from Fischer Scientific (USA). Polypropylene (PP) and polymethyl methacrylate (PMMA) were purchased from Interstate Plastics (USA). Porcine skin was purchased from a local grocery store (Irvine, CA, USA). Sea water was obtained from a local beach (Huntington Beach, CA, USA).

Instrumentation: ¹H and ²⁹Si NMR spectra were recorded on a Bruker AVANCE 600 spectrometer. Matrix-assisted laser desorption/ionization time-of-flight mass (MALDI-TOF-MS) spectrometry was performed using a-cyano-4-hydroxycinnamic acid (CHCA) as a matrix on a Bruker Daltonics Autoflex™ Speed MALDI-TOF/TOF spectrometer. Fourier-transform infrared spectra (FT-IR) were recorded on a Jasco 4700 FT-IR spectrometer. Electronic absorption spectra and fluorescence spectra were recorded on a BioTek™ Synergy™ H1 Hybrid Multi-Mode microplate reader. Optical and fluorescence microscope images were obtained on an RVL-100-M model ECHO Revolve fluorescence microscope at 60× magnification with a phase-contrast objective. Confocal laser scanning microscopy (CLSM) and fluorescence recovery after photobleaching (FRAP) experiments were performed on a Carl Zeiss 780 Laser Scanning Microscope running on Zen 2012 software, using a Plan-Apochromat 40×/1.3 Oil DIC immersion objective lens. Rheology measurements were performed on a Discovery series HR-2 hybrid rheometer from TA instruments with a 25 mm diameter parallel plate geometry. Linear rheological data were acquired at 5% strain amplitude (25° C.). Tensile testing was performed on Instron 3365 Universal Testing System with 500 N load capacity. THORLABS LED UV curing system (CS20K2, 365 nm) was used for photo-curing experiments. Static contact angles were measured on a ramé-hart Model 90 contact angle goniometer (ramé-hart, Succasunna, NJ).

Elastic liquid droplet (END) network: To an aqueous solution of SBMA (1.06 g, 3.8 mmol, dissolved in 800 mg distilled water), 0.188 g (0.76 mmol) of TMeOSMA and concentrated HCl (12.1 M, 30 μL) were added. The sample containing vial was sealed with a rubber septum, prior to vigorous bubbling with N₂ for 10 minutes. The resulting mixture was placed on the bench top, affording an elastic gel after 16 hours. Factors that were found to affect the outcome include contaminated Schlenk line (incomplete inert environment) and water impurity of the reagents due to their hygroscopic nature.

Diagram: To an aqueous solution of zwitterionic precursor (SBMA, CBMA, or PCMA), TMeOSMA and the concentrated HCl (12.1 M, 30 μL) were added as shown in the table. The sample containing vial was sealed with a rubber septum, prior to vigorous bubbling with N₂ for 10 minutes. The amount of distilled water or an aqueous solution of NaCl (0.5-5 M) was 5, 3, 2, 1, and 0.8 g to afford the total concentration of 5.7, 4.6, 2.3, 1.5, and 0.9 molality (m).

| Zwitterionic precursor/ | Zwitterionic precursor | TMeOSMA |
|---|---|---|
| TMeOSMA | (mmol) | (mmol) |
| 50 | 4.47 | 0.09 |
| 31 | 4.41 | 0.14 |
| 22 | 4.36 | 0.2 |
| 10 | 4.15 | 0.41 |
| 5 | 3.8 | 0.76 |
| 1 | 2.28 | 2.28 |
| 0.2 | 0.76 | 3.8 |

Fluorescence recovery after photobleaching experiment: To an aqueous solution of SBMA (1.06 g, 3.8 mmol, dissolved in 800 mg distilled water), 0.188 g (0.76 mmol) of TMeOSMA, concentrated HCl (12.1 M, 30 μL) and Nile red (2 μM) were added. The sample containing vial was sealed with a rubber septum, prior to vigorous bubbling with N₂ for 10 minutes. 5 μL of the resulting mixture was taken and subjected to confocal laser scanning spectroscopy (λₑₓ=515 nm, λₑₘ=550-630 nm). Using 100% intensity of excitation at 515 nm, the center of a droplet was photobleached. Fluorescence recovery patterns were recorded with a 968.15 ms frame.

Tensile strength measurement: To an aqueous solution of SBMA (1.06 g, 3.8 mmol, dissolved in 800 mg distilled water), 0.188 g (0.76 mmol) of TMeOSMA and concentrated HCl (12.1 M, 30 μL) were added. The vial was sealed with a rubber septum, prior to vigorous bubbling with N2 for 10 minutes. 300 μL of the resulting mixture was taken, mixed with an aqueous of LAP solution (final concentration: 2.88 mM), deposited on Teflon dog-bone mold (gauge length=10 mm, overall length=35 mm, thickness=1 mm), and was subjected to UV-irradiation for 2 minutes at a distance of 3.85 cm (THORLABS LED UV curing system, CS20K2, 365 nm). The photo-cured sample was loaded on the tensile testing instrument and pulled it apart until it broke with a 10 mm/mm strain rate. For the acid treatment experiment, the photo-cured sample at 0 h was soaked in an aqueous solution of HCl (pH=3) for 3 hours. The sample was taken out, dried with Kimwipes, loaded on the tensile testing instrument, and pulled apart until it broke with a 10 mm/mm strain rate.

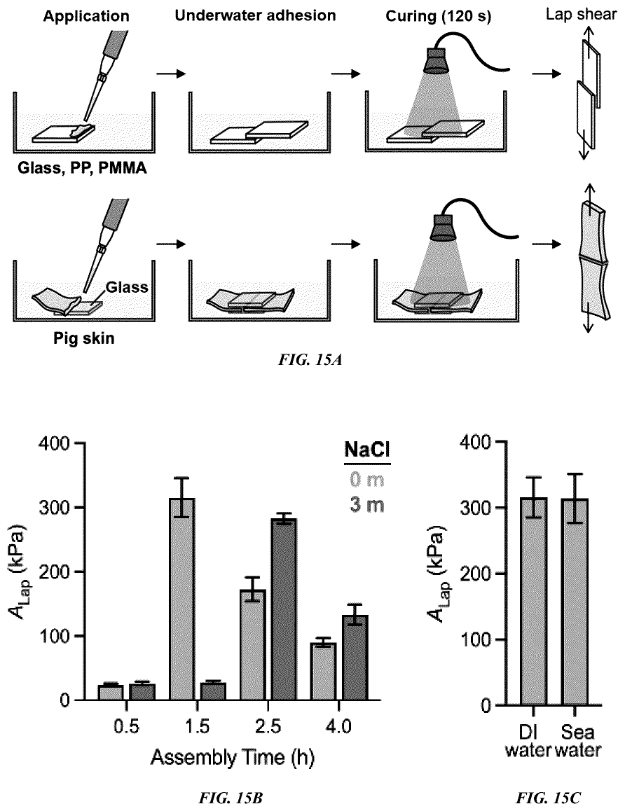

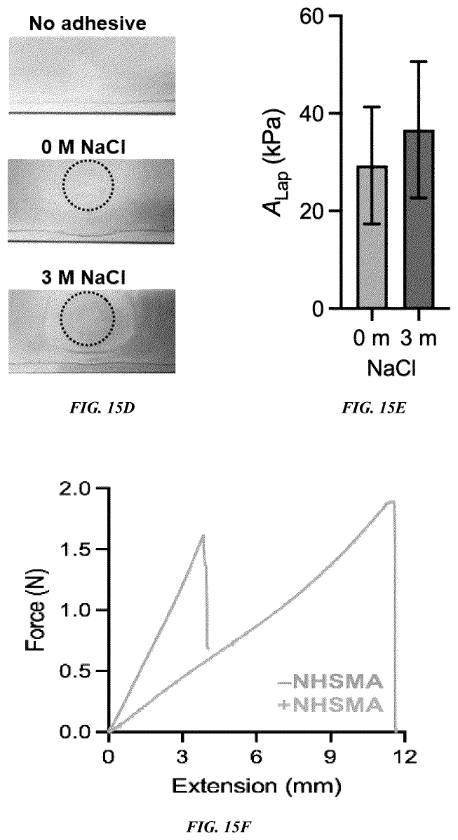

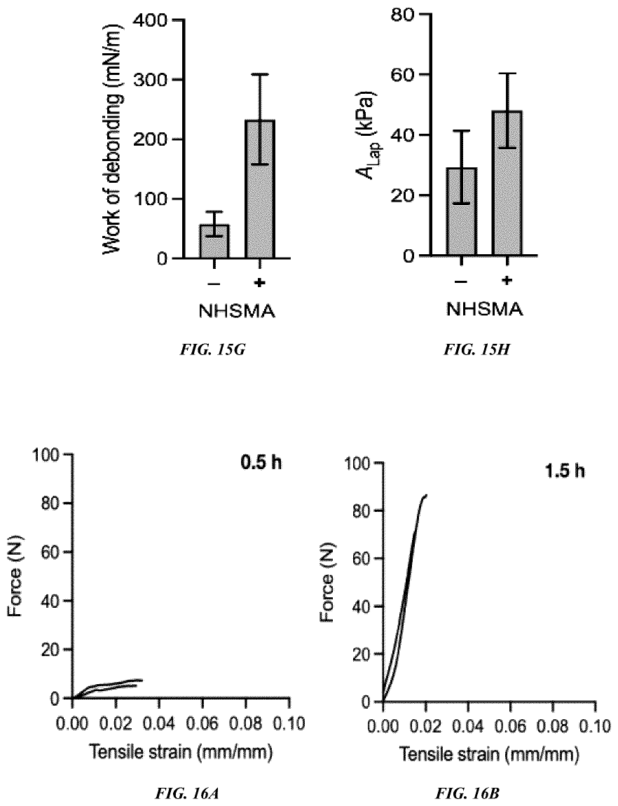

Adhesion lap shear measurement. Sample preparation: To an aqueous solution of SBMA (1.06 g, 3.8 mmol, dissolved in 800 mg distilled water), 0.188 g (0.76 mmol) of TMeOSMA and concentrated HCl (12.1 M, 30 μL) were added. The sample containing vial was sealed with a rubber septum, prior to vigorous bubbling with N₂ for 10 minutes. 300 μL of the resulting mixture was taken, mixed with an aqueous LAP solution (final concentration: 2.88 mM), and deposited on a substrate (glass, PMMA, and PP, adhesion site dimension: 25 mm×10 mm) underwater (distilled water or seawater). According to the method described in FIG. 15A, the other substrate was placed onto it. For porcine skin substrates, the mixture was deposited in the cross-sectional side cut by a razor (adhesion site dimension: 1.5 mm×20 mm), and glass support was placed on the adhesion part during the 2-min UV curing step. The glass and plastic samples were held together with hands while being subjected to UV light for 2 minutes. Adhered substrates were taken out from the water, loaded onto the tensile testing instrument, and pulled apart until they broke with a 3 mm/mm strain rate. The mixtures with less than 1 hour of assembly time are fluidic and diffused out when deposited on substrates (FIG. 17).

Sample preparation with NHSMA: To an aqueous solution of SBMA (1.06 g, 3.8 mmol, dissolved in 800 mg distilled water), 0.188 g (0.76 mmol) of TMeOSMA and concentrated HCl (12.1 M, 30 μL) were added. The sample containing vial was sealed with a rubber septum, prior to vigorous bubbling with N₂ for 10 minutes. After adding 9.3 μL of NHSMA and aqueous LAP solution (final concentration: 2.88 mM), the resulting mixture was deposited in the cross-sectional side of porcine skin substrates cut by razor (adhesion site dimension: 1.5 mm×20 mm). Glass support was placed on the adhesion part during the 2-min UV curing step. Adhered substrates were taken out from the water, loaded onto the tensile testing instrument, and pulled apart until they broke with a 3 mm/mm strain rate.

### Synthesis of FAM-TMeOSMA.

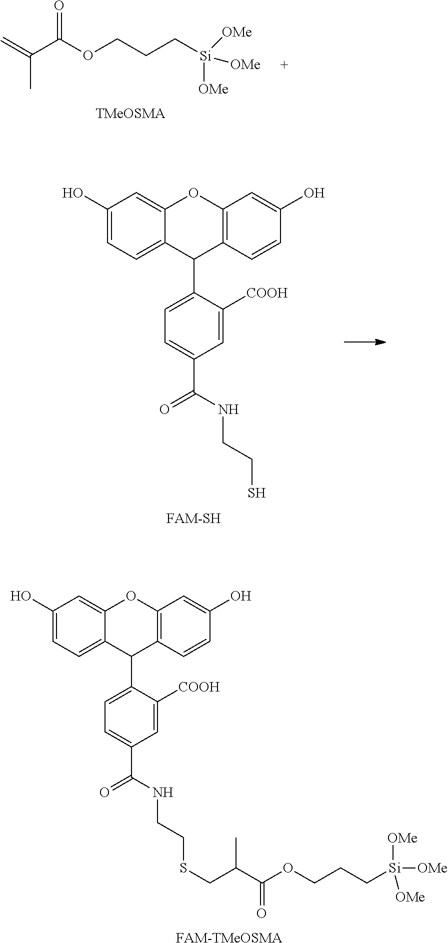

To a DMSO (20 μL) solution of FAM-SH (5 mg, 0.01 mmol), TMeOSMA (2.98 mg, 0.012 mmol) and DMAP (0.006 mmol) were added, and the mixture was stirred under nitrogen overnight. The reaction mixture was evaporated to dryness under reduced pressure and precipitated in DCM, affording FAM-TMeOSMA as a yellow powder. MALDI-TOF-MS (CHCA): m/z found: 724.3 [M+K]⁺ (calc'd 724.1).

### Synthesis of FAM-SBMA.

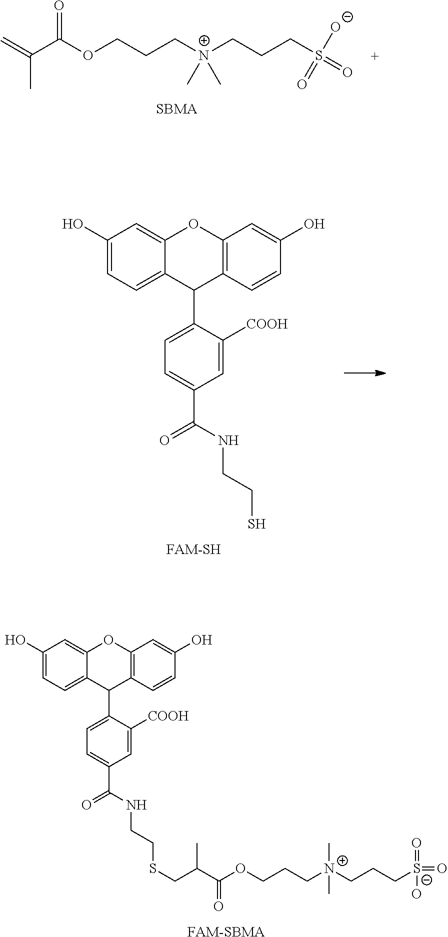

To a DMSO/water (v/v=1/1, 20 μL) solution of FAM-SH (5 mg, 0.01 mmol), SBMA (2.74 mg, 0.012 mmol) and DMAP (0.006 mmol) were added, and the mixture was stirred under nitrogen overnight. The reaction mixture was evaporated to dryness under reduced pressure, precipitated in DCM, dried, redissolved in water, and centrifuged. The supernatant was collected and lyophilized, affording FAM-SBMA as a yellow powder. FAM dye exists in equilibrium between open and closed (lactone ring) forms. MALDI-TOF-MS (CHCA): m/z found: 871.91 [M+2DMSO+H]⁺ (calc'd 871.23 based on the closed form of FAM).

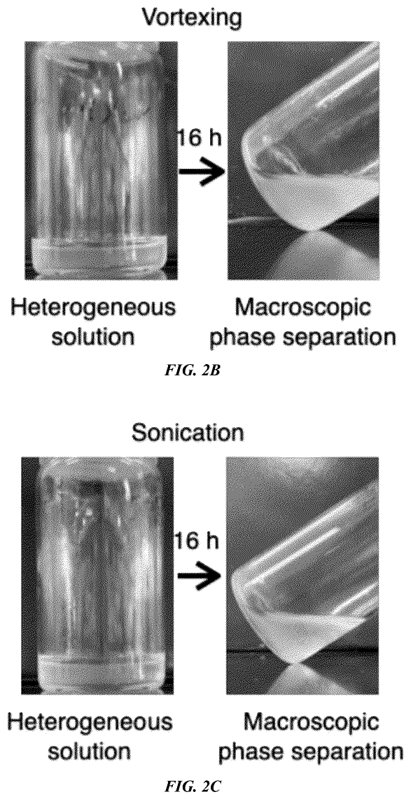

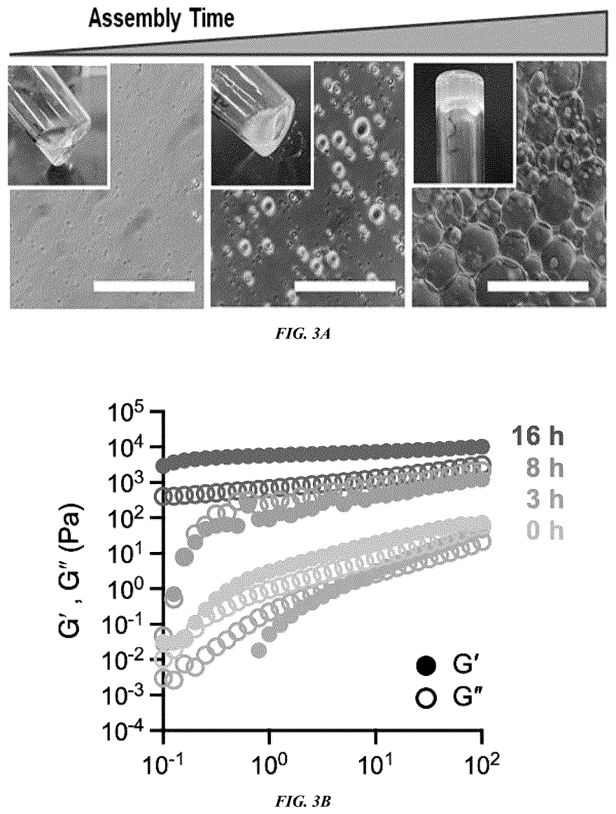

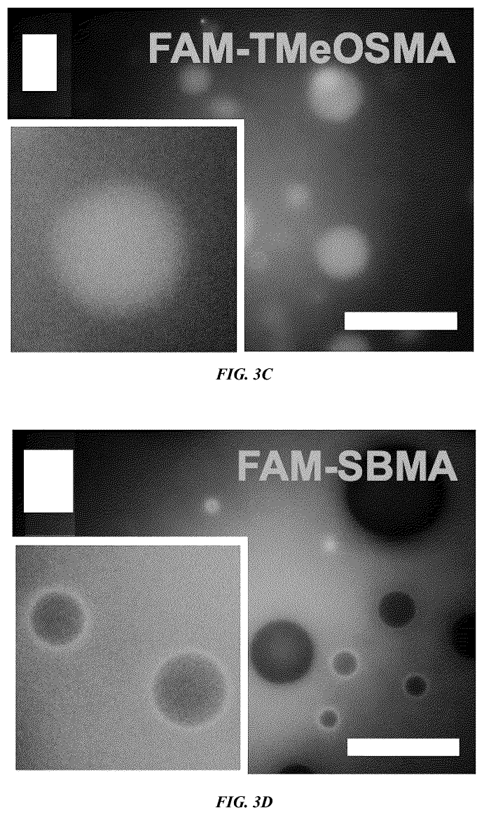

Formation of an elastic network of compartments via LLPS. The small molecule liquid precursors, TMeOSMA and SBMA, are typically immiscible (see FIG. 2). Serendipitously it was discovered that bubbling N₂ (g) into an aqueous mixture of 0.95 molal concentration (m, solute in mol per solvent in kg) of TMeOSMA and 4.75 m SBMA under acidic conditions (DI water, pH 3) turns the two immiscible phases into one homogeneous phase in 10 min. Importantly, this mixture does not contain any photoinitiators that may trigger radical polymerization at the methacrylate moiety. This clear solution over time undergoes LLPS characterized by micron-sized compartment formation and increased macroscopic turbidity, and after 16 hours yields a turbid gel comprising a network of compartments (see FIG. 3A). The inside of these compartments shows a dark contrast under phase contrast microscopy (see FIG. 3A), different from typical silica particles showing a bright contrast due to their high refractive index. The same mixture bubbled with air also becomes transparent after 10 min but undergoes macroscopic phase separation within 8 h (see FIG. 2). Linear rheological measurement of this mixture upon changing assembly time showed a gradual change from viscoelastic liquid to viscoelastic solid, accompanied by orders of magnitude changes in stiffness (see FIG. 3B).

It was postulated that the relatively more hydrophobic TMeOSMA in an aqueous mixture constitutes a compartment, and zwitterionic SBMA plays the role of surfactant at the interface, similar to surfactant-mediated stabilization of coacervates in other studies. Fluorescein amidite (FAM) thiols were conjugated to each liquid precursor through a Michael addition reaction at the methacrylate part. FAM conjugated TMeOSMA (FAM-TMeOSMA) localized inside the compartments (see FIG. 3C), and FAM-conjugated SBMA (FAMSBMA) was observed outside and at the boundary of the compartments (see FIG. 3D). The sulfobetaine motif exhibits an intermolecular zwitterionic self-association—an electrostatic locking effect, it was speculated that SBMA on the compartment surface forms interlocked structures through electrostatic interactions and kinetically stabilizes the network. Similar to LLPS, there is an energetic drive for compartments to grow in size, but once the surface SBMA molecules start to form electrostatically interlocked structures, they stop growing. The temperature was increased at which LLPS proceeds to 80° C. and obtained a turbid gel without compartment formation (see FIG. 4), indicating that the elastic network of compartments is indeed a kinetic product.

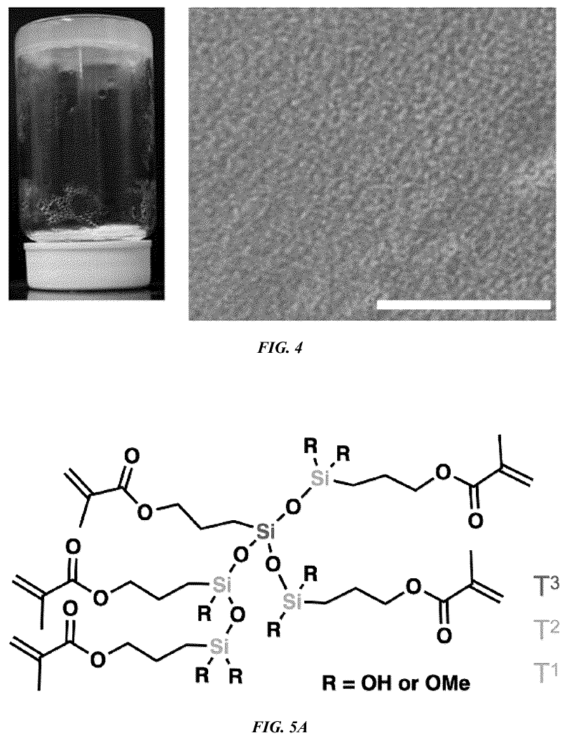

Hydrolysis and silanol condensation occur within liquid droplets. The trimethoxysilyl group from TMeOSMA is known to undergo hydrolysis and condensation reactions under mildly acidic conditions. It was hypothesized that upon mixing the two immiscible liquid precursors and bubbling N₂ under acid conditions, hydrolysis of the trimethoxysilyl group into silanol would occur, making the two liquids miscible, resulting in a homogeneous mixture. ²⁹Si NMR spectroscopy over time revealed dynamic changes in TMeOSMA (see FIG. 5A). Silicon atoms are labeled with T, and the superscript number represents the number of adjacent siloxane bonds. Even at the beginning of the LLPS process after 10-minute of N2 bubbling (0 h), peaks from the lower degree of the condensed form of silanol (T¹ and T²) were visible along with a noticeable silanol peak (T°, Si-OH), indicating that TMeOSMA has gone through acid-mediated hydrolysis and lower degree of condensation during the N₂ bubbling step. As droplets formed and grew, both trimethoxysilyl group (T⁰, Si-OMe) from the precursor and its hydrolyzed form silanol (T⁰, Si-OH) disappeared. At the same time, peaks from the siloxane oligomers (T¹, T², and T³) gradually increased. The degree of condensation was calculated through the integration of peaks (see FIG. 5B). Condensation rapidly proceeded within the first 2 hours and gradually reached up to 95%. It was noted that despite the high degree of condensation at the endpoint, most silicon species were at T¹ or T². Fourier-transform infrared spectroscopy (FT-IR) of these mixtures also showed that the peak from the Si-OMe stretch started to disappear at the beginning of the LLPS after N₂ bubbling, and a new peak corresponding to siloxane bonds emerged (see FIG. 5C). Taken together, the SBMA and TMeOSMA mixture becomes miscible upon N₂ bubbling under an acidic condition due to partial hydrolysis of TMeOSMA into silanol, which is more hydrophilic. At the same time, further condensation into macromolecular siloxane triggers LLPS in the course of 16 h.

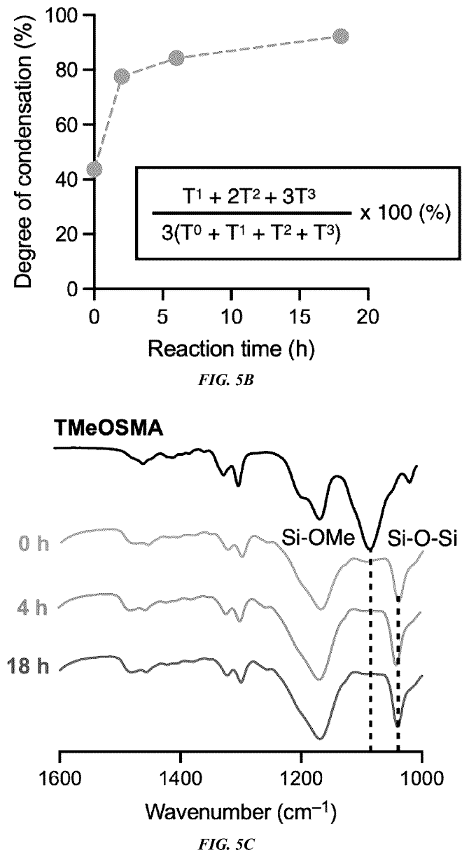

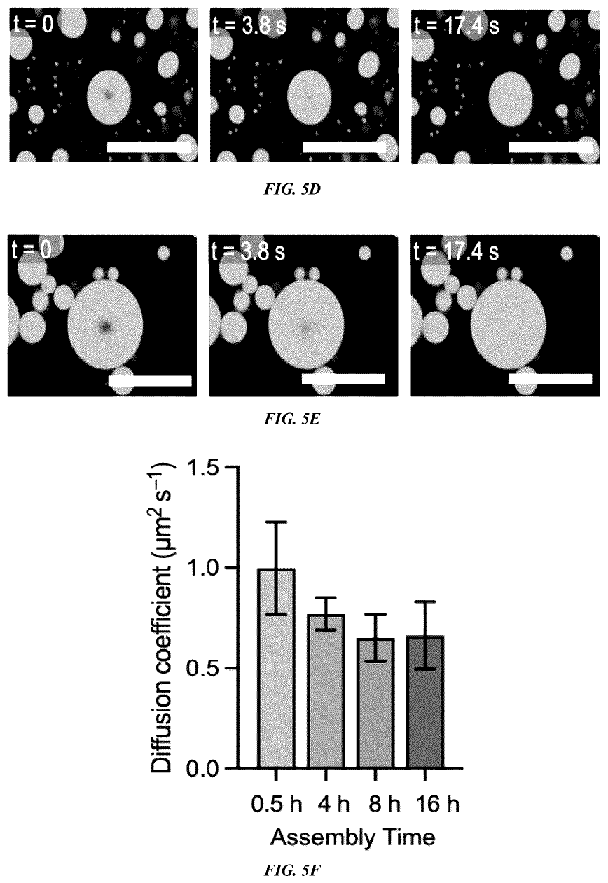

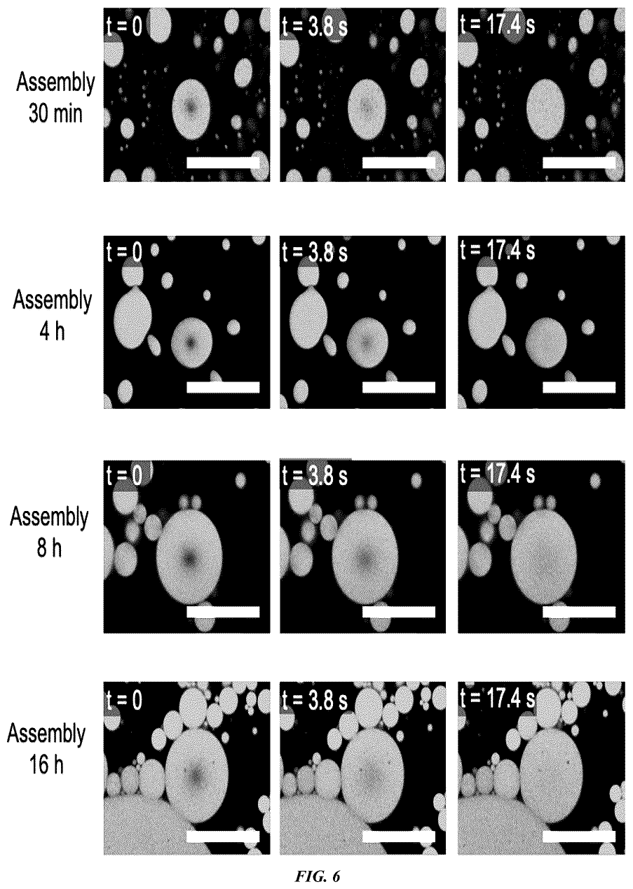

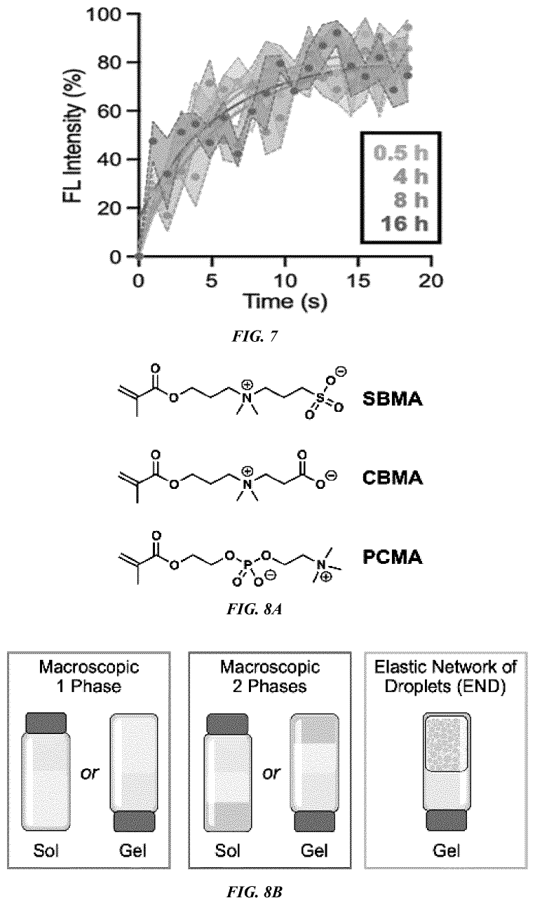

Fluorescence recovery after photobleaching (FRAP) experiments were performed to determine the fluidity and diffusion characteristics of the droplets, using Nile red as a probe molecule (see FIGS. 5D-F). Upon photobleaching (λₑₓ=515 nm, 100% intensity), the fluorescence of droplets recovered up to 70-90% of its initial value in 20 s for all of the time points (see FIGS. 6 and 7). The diffusion coefficients of the dye inside the droplet were determined from the FRAP results (see FIG. 5F and FIGS. 6 and 7) revealing a slight decreasing trend in diffusion coefficient as the assembly time increases. Overall, the fluidic nature of the siloxane droplets is maintained through the LLPS process and the resulting elastic network of droplets (END) phase.

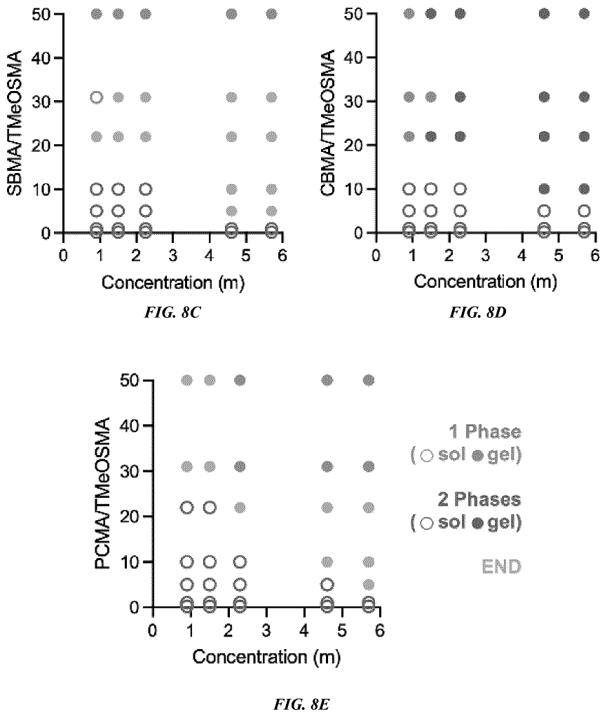

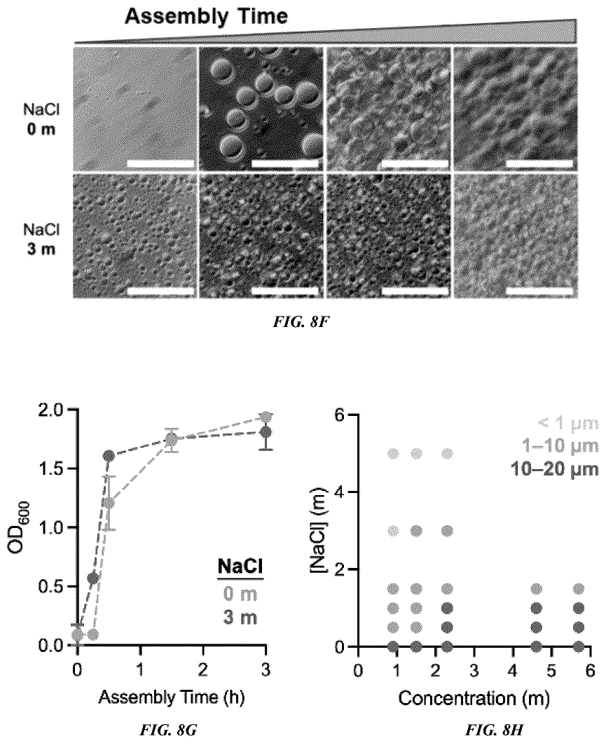

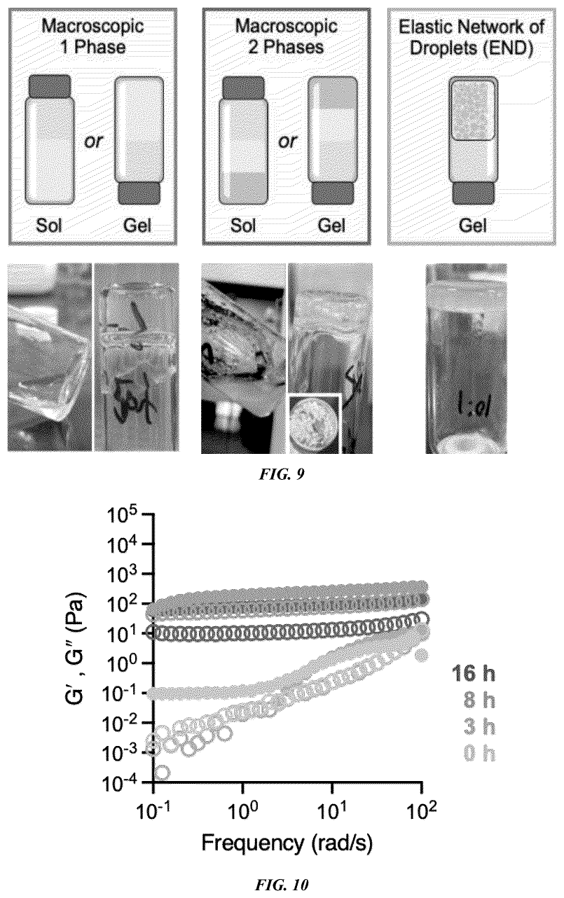

Effect of molecular structures and chemical environment on the END phase formation. To test whether the electrostatic locking effect of SBMA is responsible for END phase formation, the effect of molecular structures and compositions on phase behaviors were investigated with a few different zwitterionic monomers; SBMA, 3-3-[[2-(methacryloyloxy)ethyl]dimethylammonio]propionate (carboxybetaine methacrylate, CBMA), and 2-methacryloyloxyethyl phosphorylcholine (phosphorylcholine methacrylate, PCMA) (see FIG. 8A). The ratio of TMeOSMA/zwitterion and total concentrations of small molecules were varied, and the outcomes were recorded based on the macroscopic phases and sol/gel states (see FIG. 8B and FIG. 9). TMeOSMA and SBMA mixtures at low ratios of SBMA resulted in solutions with macroscopically segregated two phases, and at the highest ratio of 50, samples with all tested concentrations became macroscopically homogeneous gels in 1 hour (see FIG. 8C). The END phase occurs at the boundary of these two phases. It is suspected that when the number of SBMA molecules in a system is not enough to stabilize hydrophobic droplets generated from TMeOSMA, these droplets eventually coalesce into a macroscopic solution. On the other hand, at the highest ratio of SBMA/TMeOSMA, excess SBMA may homogeneously disperse TMeOSMA species and their hydrolyzed forms. A different zwitterionic monomer, CBMA, has been shown to exhibit a lower degree of self-association due to a mismatch in charge density (ammonium cation: 3.0 e/nm³, sulfate anion: −4.5 e/nm³, and carboxylate anion: −5.3 e/nm³). In contrast to the mixtures of SBMA, CBMA mixed with TMeOSMA did not result in the END phase in all tested ratios and concentrations (see FIG. 8D). Most combinations yielded macroscopic 2 phases, either in gels or solutions, and the homogeneous single phase was only observed in lower total concentrations and high CBMA ratios. This result highlights the importance of the zwitterion's self-association in arriving at and stabilizing the END phase after the LLPS process. PCMA was also tested, which has been shown to bear a similar charge density to SBMA (ammonium cation: 3.0 e/nm³, phosphate anion: −3.0 e/nm³). Similar to SBMA, PCMA and TMeOSMA mixtures yielded the END phase at the phase boundary (see FIG. 8E, and FIG. 10).

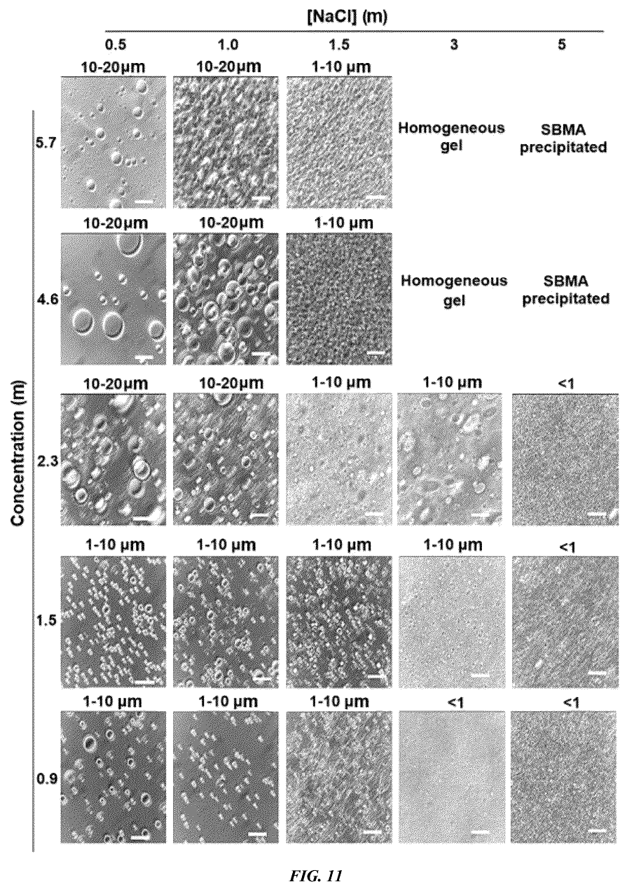

It was further tested whether adding salt, which has been shown to affect electrostatic interactions between charged species, changes the phase behavior of the system. The mixture of SBMA/TMeOSMA (ratio=5, total concentration=5.7 m) in the presence of 3 m NaCl formed smaller droplets than in the absence of NaCl (see FIG. 8F). Interestingly, in the presence of the salt, droplet formation occurred slightly faster than in the absence of the salt, based on turbidity (OD₆₀₀) measurements at the early time points (see FIG. 8G). Both samples arrived at the END phase after 16 hours. The NaCl concentration and the total concentration of the methacrylate species was systematically varied at a fixed monomer ratio SBMA/TMeOSMA of 5 (see FIG. 8H and FIG. 11). Similar to the observations in FIG. 8F, higher concentrations of NaCl result in smaller-sized droplets in all tested concentrations. Data at high concentrations of NaCl and methacrylate species were not able to be collected because they yielded precipitation or homogeneous gels (see FIG. 11). These results show that electrostatic interaction between zwitterionic molecules on the droplet surface determines the energetic landscape and phase behaviors.

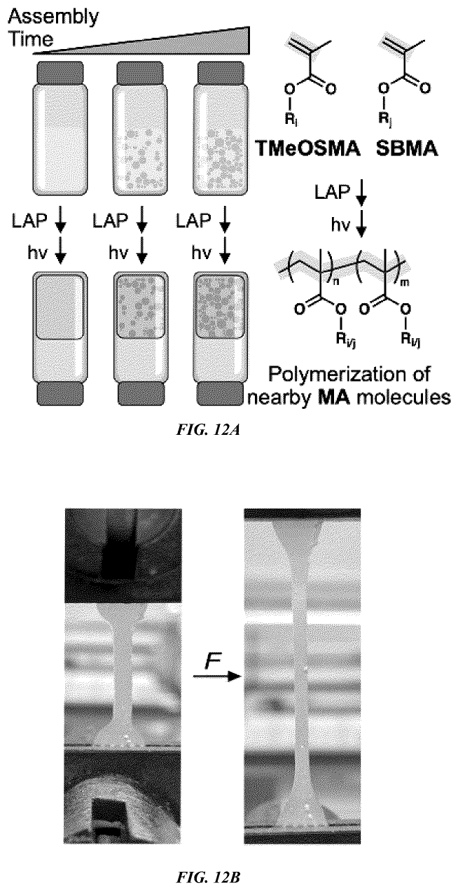

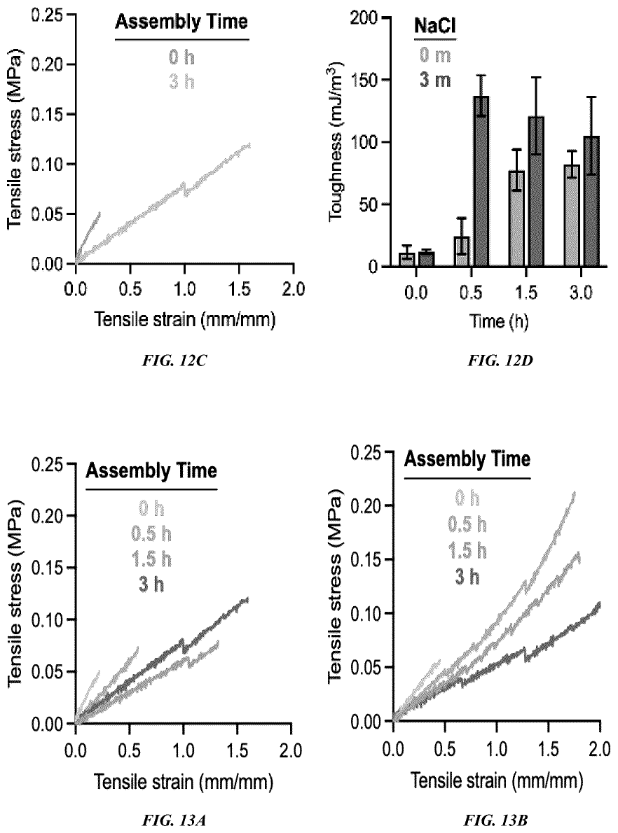

Hierarchical microstructures increase toughness and stretchability. As the self-assembled droplet mixtures have unreacted methacrylate handles, studies were conducted on how the hierarchical structure impacts the overall mechanical performance of materials. Since the droplet size is a function of NaCl concentration and assembly time (see FIG. 8F-H), these parameters were varied in the samples. Samples are mixed with a photoinitiator, lithium phenyl-2,4,6-trimethylbenzoylphosphinate (LAP), at various stages of assembly and subjected to photo-crosslinking with 365 nm LED light (see FIG. 12A). The hierarchical morphology of each state is fixed by polymerizing the methacrylate part of the two small molecule precursors. Self-assembled structures were only compared within the first 4 hours of assembly because introducing LAP to the END phase resulted in heterogeneous material due to the non-homogeneous incorporation of the photoinitiator.

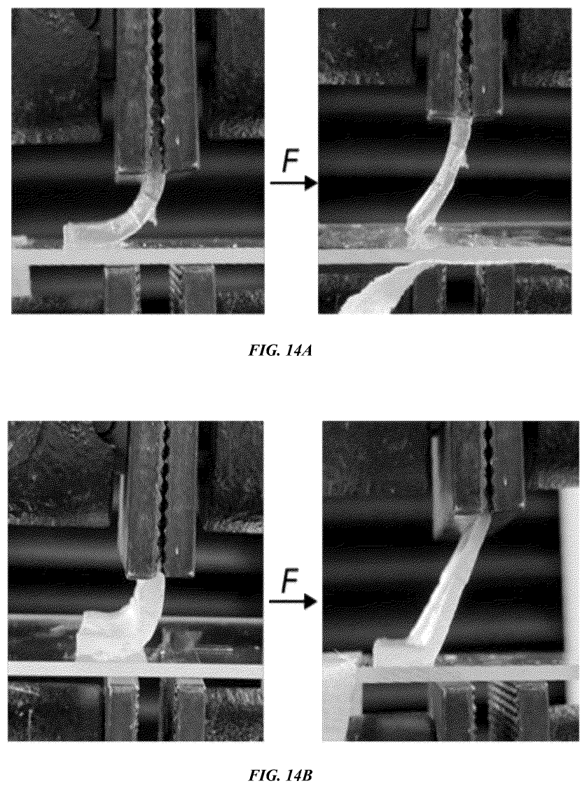

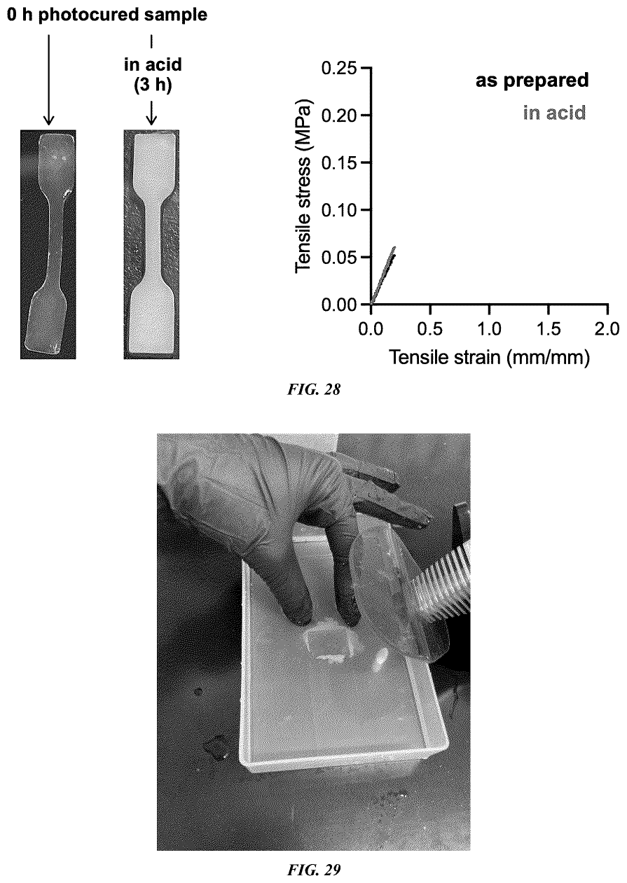

Tensile testing of the fixed samples showed that hierarchical microstructures make them less brittle and more stretchable than homogeneous mixtures at the beginning (see FIG. 12B). There was a clear increase in toughness and failure strain of the materials as the assembly time increased (see FIG. 12C). Similarly, samples prepared with 3 m NaCl showed an increase in stretchability, toughness, and failure strain with longer assembly time (see FIG. 13). The assembly time was systematically varied in the absence and presence of 3 m NaCl and compared the resulting materials' toughness (see FIG. 12D). Mixtures at 0 h without droplet structures yielded brittle materials in both cases. At 0.5 h, the 3 m NaCl mixture with a significant droplet formation showed an 11.4-fold increase in toughness compared to 0 h. The differences in stiffness were correlated with the extent of droplet formation prior to the photo-polymerization (see FIG. 8G). However, any obvious effect from the droplet size was not observed. While both systems show a similar extent of droplet formation based on turbidity at a 3-h assembly time, the stiffness of the resulting materials was similar. Interestingly, longer assembly time also increased macroscopic adhesion to glass substrates, which may have resulted from the structuring of assembled SBMA molecules on the droplet and material surface (see FIG. 14). To isolate the effect of hierarchical structures from silanol condensation, photo-crosslinked samples at 0 h were prepared and then incubated in an acidic environment for 3 h to promote postcuring silanol condensation. The resulting materials showed increased turbidity but were brittle and exhibited similar mechanical properties to pristine materials crosslinked at 0 h (see FIG. 28), indicating that hierarchical microstructure is the key to increasing toughness, stretchability, and adhesion of the materials.

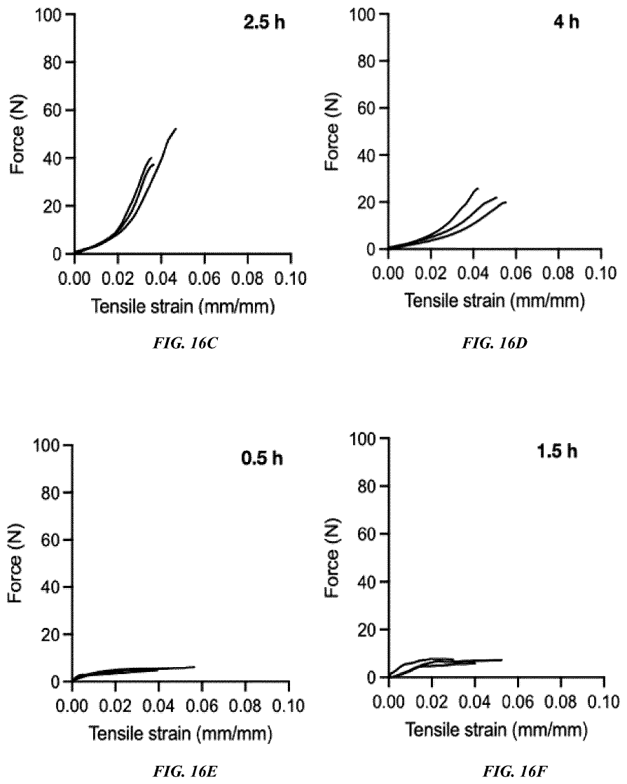

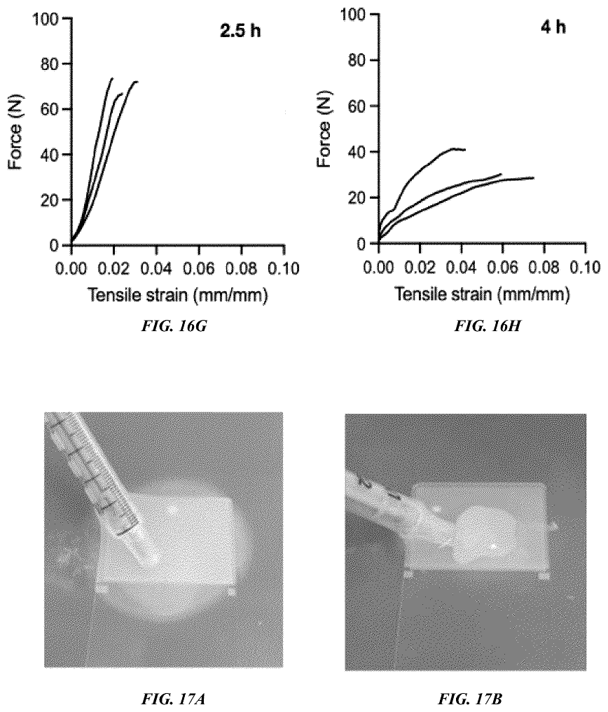

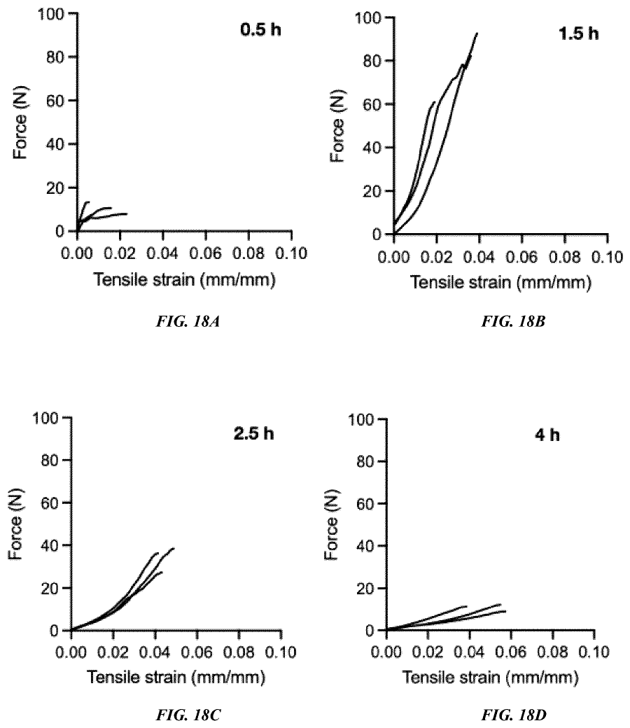

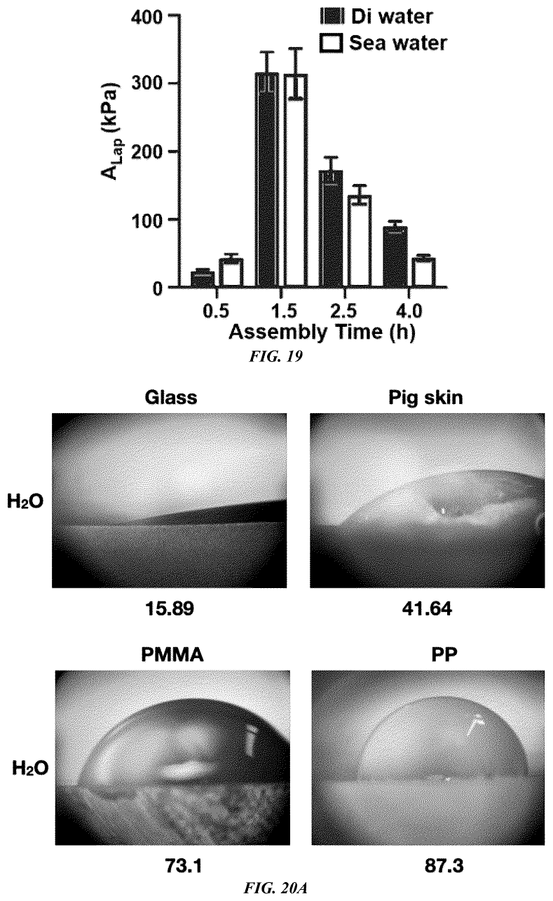

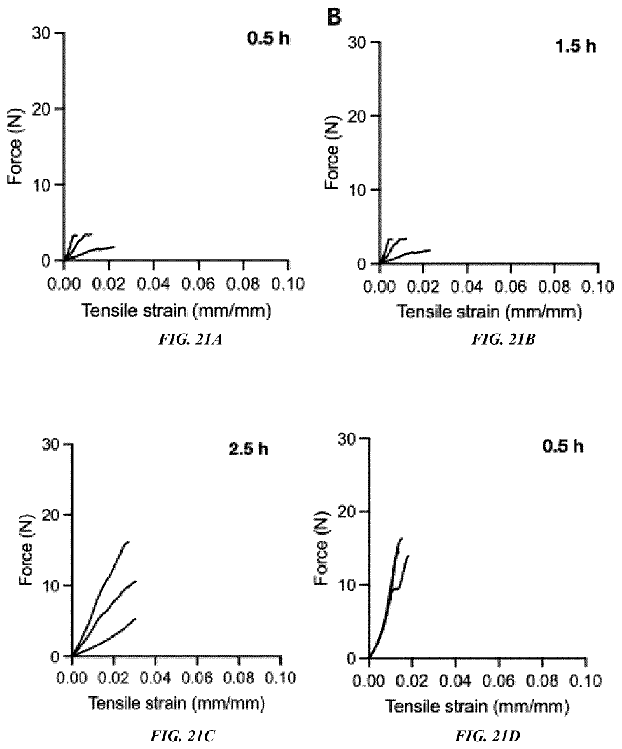

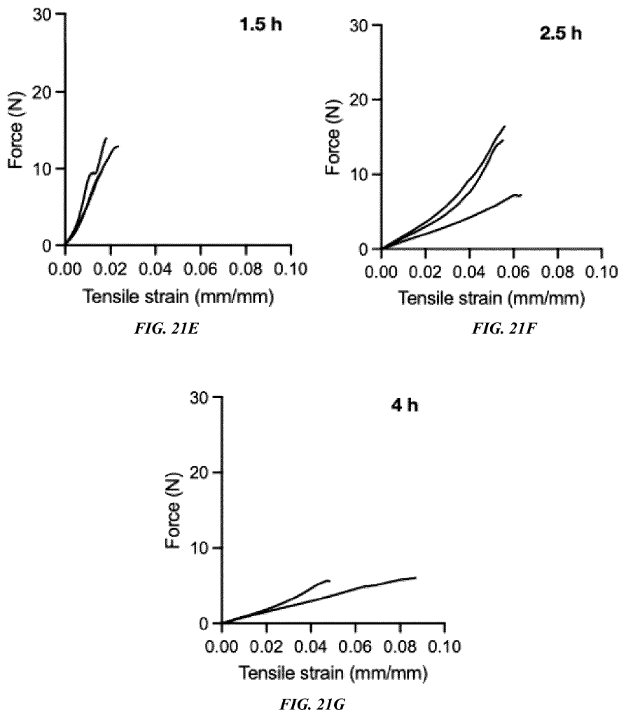

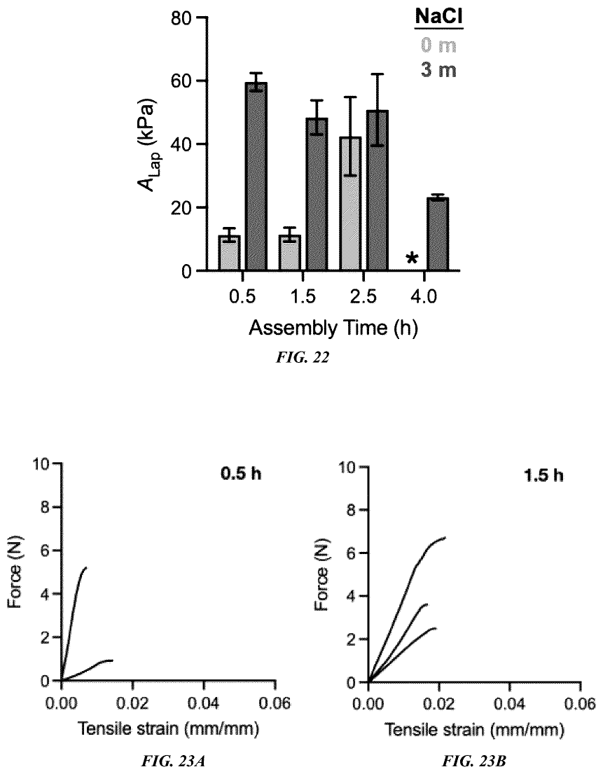

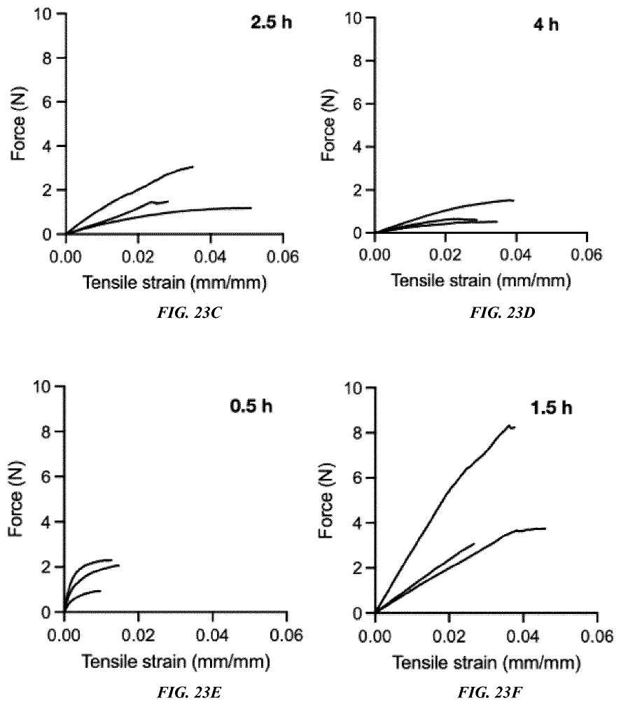

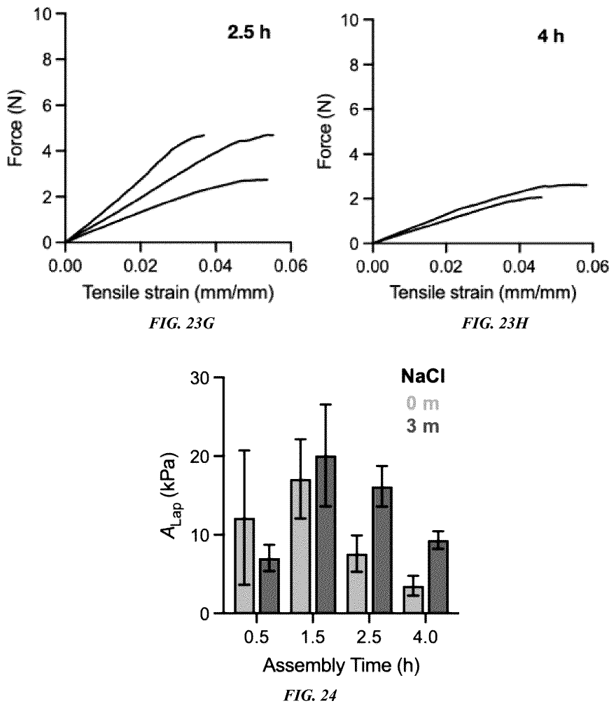

Tough and fast-acting underwater adhesives. Robust adhesion in wet environments is useful for medical and watercraft industries. Underwater adhesion is typically more challenging than in air due to the surrounding water molecules. Many synthetic underwater adhesives have been developed, but current limitations include impractical curing time (>1 h), use of organic solvents, and multi-step synthesis of macromolecules limiting scalability. Encouraged by the observed adhesion and increased toughness of the materials, their underwater adhesion behavior using various substrates was investigated. Briefly, a precursor liquid (300 μL) was applied to a mixture that underwent LLPS with a specific assembly time, on the surface of glass, PP, and PMMA substrates (10×20 mm²). After placing another substrate directly onto this area, UV irradiation (λ=365 nm) was applied for 2 minutes (see FIG. 15A, upper panel). For testing porcine skin substrates, the precursor mixture was applied to the cross-sectional side (1.5×20 mm²) cut by a razor with the help of glass support for adhesion during UV irradiation (see FIG. 15A, lower panel). Adhesion strength for glass substrates reached 314±30.5 kPa (see FIG. 15B and FIG. 16) with the precursor liquid prepared without NaCl and an assembly time of 1.5 h. It was also noted that the same mixture at the assembly time of less than 1 h tends to diffuse away from the substrates and is not deposited stably on a glass substrate (see FIG. 17). Overall, the trend of adhesion strength values did not show a direct correlation with the toughness data in FIG. 12D. This phenomenon was attributed to the additional effect of substrate interaction with the precursor mixtures. This material showed a similar adhesion strength value when prepared and cured under seawater from Huntington Beach, California (see FIG. 15C, FIG. 18, and FIG. 19). Polymethylmethacrylate (PMMA) and polypropylene (PP) were also tested, and they showed adhesion values of 60±3.8 kPa and 20±7.7 kPa, respectively (see FIGS. 21-24).

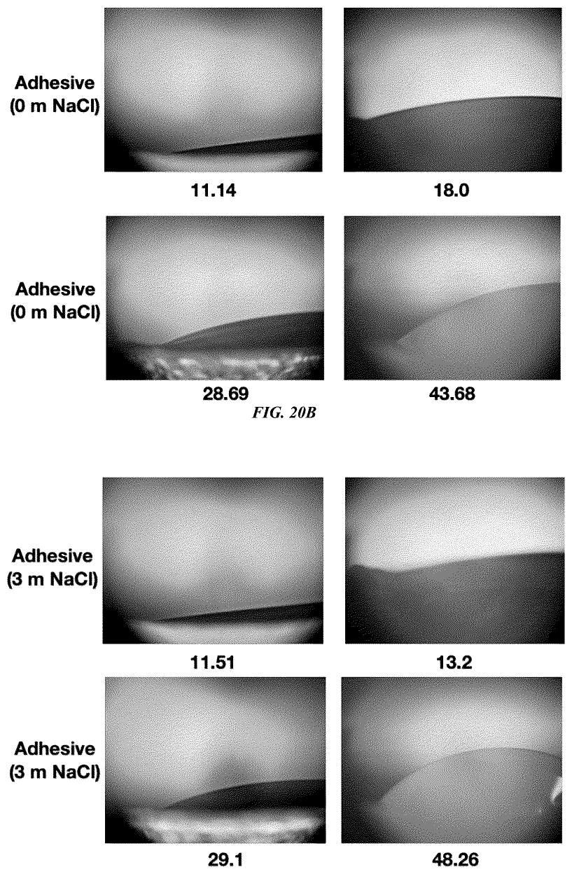

It was directly observed the interaction between precursor mixture with water on a wet glass surface to understand how these materials function as an underwater adhesive (see FIG. 15D). As soon as the precursor mixture was applied, the surrounding interfacial water was immediately repelled, further away from the substrate-precursor boundary. Contact angle measurements also revealed that substrate-precursor interaction was stronger than substrate-water interaction in all tested substrates (see FIG. 20).

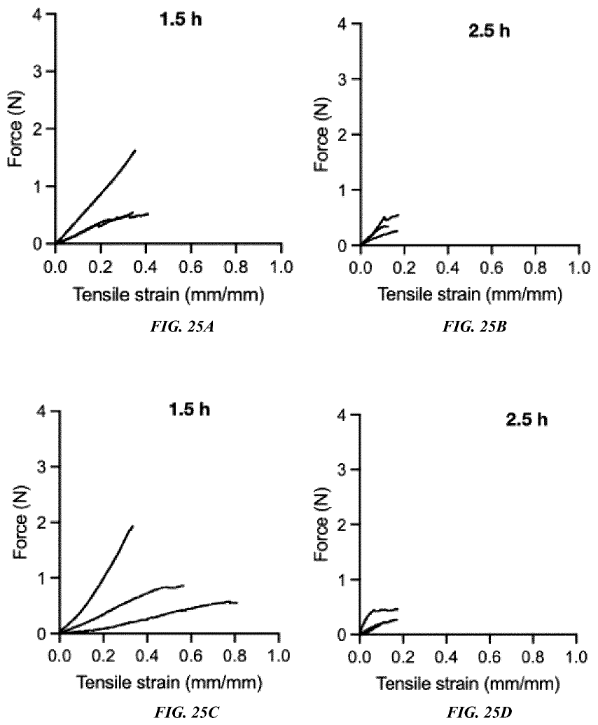

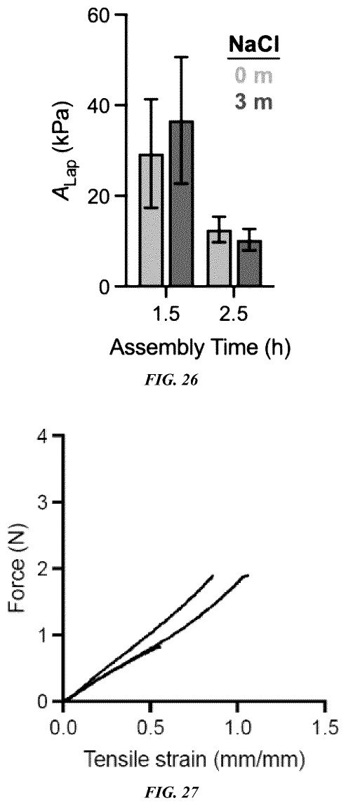

The adhesion value of this material with porcine skin substrates reached 37±17 kPa with a precursor solution in 3 m NaCl (see FIG. 15E, FIG. 25, and FIG. 26). Because porcine skin bears various nucleophilic functional groups on its surface, it was sought to suppress adhesive failure and increase overall adhesion performance by incorporating methacrylic acid N-hydroxysuccinimide ester (NHSMA). Tensile testing of the adhesives containing NHSMA (1 wt % in methacrylate species) showed increased failure strain than the ones without NHSMA (see FIG. 15F, and FIG. 27). Work of debonding (WoD) of the adhesives containing NHSMA showed a 3.7-fold increase from the one without NHSMA (see FIG. 15G). NHSMA also increased adhesion strength (see FIG. 15H). Therefore, adhesives with NHSMA showed increased ductility, which is important for the performance stability of adhesives by preventing sudden failures.

A number of embodiments have been described herein. Nevertheless, it will be understood that various modifications may be made without departing from the spirit and scope of this disclosure. Accordingly, other embodiments are within the scope of the following claims.

What is claimed is:

1. A viscoelastic material having a solid or gel-like consistency, comprising:
an elastic network of compartmentalized liquid droplets, wherein each liquid droplet has an exterior surface and inner compartment, wherein the inner compartment comprises a silyl-based compound and wherein the exterior surface comprises a zwitterion-based compound,
wherein the electrostatic interactions between the compartmentalized liquid droplets interlock the compartmentalized liquid droplets together to form a viscoelastic material having a solid or gel-like consistency.

2. The viscoelastic material of claim 1, wherein the elastic network of compartmentalized liquid droplets is a kinetic product.

3. The viscoelastic material of claim 1, wherein the silyl-based compound forms interconnected silyl-based compounds via siloxane bonds.

4. The viscoelastic material of claim 1, wherein the silyl-based compound is selected from 3-(trimethoxysilyl)propyl methacrylate, 3-(trimethoxysilyl)methyl methacrylate, 3-(trimethoxysilyl)ethyl methacrylate, 3-(trimethoxysilyl)butyl methacrylate, 3-(dimethoxy(methyl)silyl)propyl methacrylate, 3-(dimethoxy(methyl)silyl)methyl methacrylate, 3-(dimethoxy(methyl)silyl)ethyl methacrylate, 3-(dimethoxy(methyl)silyl)butyl methacrylate, 3-(dimethyl(methoxy)silyl)propyl methacrylate, 3-(dimethyl(methoxy)silyl)methyl methacrylate, 3-(dimethyl(methoxy)silyl)ethyl methacrylate, 3-(dimethyl(methoxy)silyl)butyl methacrylate, (trimethylsilyl)methacrylate, 3-[Tris(trimethylsiloxy)silyl]propyl methacrylate, bis(trimethylsiloxy)methylsilyl methacrylate, triisopropylsilyl methacrylate, and tributylsilyl methacrylate.

5. The viscoelastic material of claim 4, wherein the silyl-based compound is 3-(trimethoxysilyl)propyl methacrylate (TMeOSMA).

6. The viscoelastic material of claim 1, wherein the zwitterion-based compound is selected from 2-(methacryloyloxy)ethyl]dimethyl-(3-sulfopropyl)ammonium hydroxide, 2-(methacryloyloxy)ethyl]dimethyl-(3-sulfoethyl)ammonium hydroxide, 2-(methacryloyloxy)ethyl]dimethyl-(3-sulfobutyl)ammonium hydroxide, 3-3-[[2-(Methacryloyloxy)ethyl]dimethylammonio]propionate, 3-3-[[2-(Methacryloyloxy)ethyl]dimethylammonio]acetate, 3-3-[[2-(Methacryloyloxy)ethyl]dimethylammonio]butyrate, and 2-methacryloyloxyethyl phosphorylcholine.

7. The viscoelastic material of claim 6, wherein the zwitterion-based polymerizable compound is 2-(methacryloyloxy)ethyl]dimethyl-(3-sulfopropyl)ammonium hydroxide (SBMA).

8. The viscoelastic material of claim 1, wherein the ratio of the molality concentration of the zwitterion-based compound to the molality concentration of the silyl-based compound is from 2:1 to 50:1.

9. The viscoelastic material of claim 1, wherein the viscoelastic material further comprises a reactive monomer selected from methacrylic acid N-hydroxysuccinimide ester (NHSMA), methacrylic acid N-hydroxysuccinimide ester, N-(Hydroxymethyl)acrylamide acrylic acid, N-hydroxysuccinimide ester paraformaldehyde, glycidyl methacrlyate, and glycidyl acrylate.

10. The viscoelastic material of claim 9, wherein the viscoelastic material further comprises NHSMA.

11. The viscoelastic material of claim 1, wherein the viscoelastic material further comprises a water soluble photoinitiator selected from lithium phenyl-2,4,6-trimethylbenzoylphosphinate (LAP), sodium 4-[2-(4-morpholino)benzoyl-2-dimethylamino]butylbenzenesulphone (MBS), 2-hydroxy-1-[4-(2-hydroxyethoxy) phenyl]-2-methyl-1-propanone, BAPO-OLi, BAPO-ONa, and 2,2′-azobis[2-methyl-N-(2-hydroxyethyl) promionamide] (VA-086).

12. The viscoelastic material of claim 11, wherein the viscoelastic material further comprises LAP.

13. The viscoelastic material of claim 1, wherein the viscoelastic material further comprises a salt at a molality from 0 m to 10 m.

14. An adhesive comprising the viscoelastic material of claim 1 that is capable of adhering to a substrate that is exposed to an aqueous environment.

15. The adhesive of claim 14, wherein the aqueous environment comprises water that has from 0 ppt up to 50 parts per thousand of dissolved salts.

16. A bandage, dressing, or medical implant comprising the viscoelastic material of claim 1, wherein the viscoelastic material is capable of adhering to biological tissue.

17. The bandage, dressing, or medical implant of claim 16, wherein the biological tissue is skin tissue.

18. A process of manufacturing the viscoelastic material of claim 1, comprising:
forming a mixture by combining a silyl-based compound and an acid, to an aqueous solution comprising a zwitterion-based compound;
vigorously introducing a gas into the mixture to form a homogenous mixture;
optionally, adding a photoinitator and/or a reactive monomer to the homogenous mixture;
solidifying the homogenous mixture into a viscoelastic material having a solid or gel-like consistency, wherein the homogenous mixture spontaneously forms the viscoelastic material over a period of time.

19. The process of claim 18, wherein the silyl-based compound is 3-(trimethoxysilyl)propyl methacrylate (TMeOSMA); the zwitterion-based compound is 2-(methacryloyloxy)ethyl]dimethyl-(3-sulfopropyl)ammonium hydroxide (SBMA); the pH of the mixture is from 1 to 4; and/or the gas is nitrogen gas.

20. The process of claim 18, wherein the homogenous mixture comprises the photoiniator, or comprises the photoiniator and the reactive monomer, and the method further comprises the step of:
curing or fixing the viscoelastic material by exposing the viscoelastic material to light having a wavelength in the ultraviolet spectral range,
wherein the photoiniator is lithium phenyl-2,4,6-trimethylbenzoylphosphinate (LAP) and the reactive monomer is methacrylic acid N-hydroxysuccinimide ester (NHSMA).
